(19) United States
(12) Patent Application Publication
(10) Pub. No.: US 2023/0197277 A1
(43) Pub. Date: Jun. 22, 2023

(54) Assessment and Quantification of Imperfect dsDNA Break Repair for Cancer Diagnosis and Treatment

(71) Applicant: Board of Regents, The University of Texas System, Austin, TX (US)

(72) Inventors: Zbyszek Otwinowski, Dallas, TX (US); Dominika Borek, Dallas, TX (US); Raquel Bromberg, Dallas, TX (US)

(73) Assignee: Board of Regents, The University of Texas System, Austin, TX (US)

(21) Appl. No.: 18/168,565

(22) Filed: Feb. 13, 2023

Related U.S. Application Data
(63) Continuation of application No. PCT/US2021/049060, filed on Sep. 3, 2021.
(60) Provisional application No. 63/074,371, filed on Sep. 3, 2020.

(51) Int. Cl. G16H 50/20; G16B 30/00; G16B 40/20

(52) U.S. Cl. CPC G16H 50/20; G16B 30/00; G16B 40/20

(57) ABSTRACT

Methods and devices for the prevention, treatment and diagnosis of cancer include assessing and quantifying imperfect dsDNA break repair. The methods may include determining a deletion signal for a DNA-containing sample of a subject, wherein the deletion signal comprises distributions of deletions (frequencies) of deletions with microhomologies of different lengths at the deletion sites in a DNA sequence or genome of the subject or sample thereof. The method may further include decomposing the deletion signal into components corresponding to changes arising from: (1) DNA repair processes, (2) systematic effects due to mapping personal deletion variants to reference genomes, and (3) false positive deletions generated during sample preparation, sequencing, and analysis, and quantifying these components to produce mutational signatures of defective HRR.

### CROSS REFERENCE TO RELATED APPLICATION

This application claims the benefit of U.S. Provisional Application No. 63/074,371 filed on Sep. 3, 2020, the disclosure of which is hereby incorporated by reference in its entirety.

### BACKGROUND

The present inventive concept is directed to methods and devices for the prevention, treatment, and diagnosis of cancer, among other uses. The present inventive concept is also directed to methods for the treatment and diagnosis of cancer that include assessing and quantifying imperfect double strand DNA (dsDNA) break repair as well as devices for the assessment and quantification of imperfect dsDNA break repair.

In many cancers, such as breast, ovarian, prostate, and pancreatic cancers, cancer cells have defective dsDNA break repair due to some dysfunction in the homologous recombination repair (HRR) pathways. The main pathways involved in dsDNA break repair are HRR and non-homologous end joining (NHEJ) and there are also alternative pathways, e.g., single-strand annealing (SSA). Among dsDNA break repair pathways, HRR is the cell's highest fidelity method of repairing double-stranded DNA breaks; however, HRR deficiency e.g., due to mutations in BRCA1 and/or BRCA2, redirects DNA repair to the more error-prone mechanisms, e.g. NHEJ. These mechanisms may introduce errors that are not simple substitutions. These errors are referred to as DNA scars or genomic scars. The genomic scars have characteristics distinct from replication errors and have a complex sequence signatures (e.g. multiple substitutions, an indel plus a substitution, an indel in a non-repetitive element). The most frequent changes are deletions. The details of mechanisms of DNA damage repair are not well understood.

Dysfunction of HRR in cancer cells creates vulnerabilities that can be used in treatment. The identification of tumors with HRR dysfunction is clinically important, as such tumors are sensitive to certain classes of drugs including, but not limited to poly [ADP-ribose] polymerase (PARP) inhibitors. Clonally amplified deletions resulting from defective HRR have been detected in cancers, in which cells have both copies of HRR-associated genes BRCA1 and BRCA2 inactivated. The PARP inhibitors are used to treat cancers with such defects.

The deletion-containing mutational signatures have been identified before in cancer tissues. However, to be included in this mutational signature, the same deletion had to be observed independently multiple times in sequencing reads, implying that the deletion was present in multiple different cells and so it was clonally amplified in the tissue fragment before the tissue was sequenced. However, well before a deletion is observed some arbitrary number of times in the results of sequencing, the defective HRR may generate many more deletions that happen only once or twice in all cells in an organism or in an organ. Because these somatic deletions are distributed randomly and sparsely in the genomic DNA, there are currently no efficient methods to identify these deletions (i.e., deletions that are not clonally amplified) before some very small number of them becomes amplified, e.g. by cancer growth. Additional methods for assessing and quantifying imperfect dsDNA break repair as well as devices for the assessment and quantification of imperfect dsDNA break repair are desirable. Further, additional methods and devices for the prevention, treatment, and diagnosis of cancer, are needed in the field.

### SUMMARY OF THE INVENTION

The present inventive concept is directed to methods and devices for the prevention, treatment, and diagnosis of cancer, among other uses.

Aspects of the present disclosure provide methods of quantifying the deletions resulting from imperfect DNA repair from a DNA-containing sample of a subject. In some embodiments, methods herein may comprise: providing sequence data, comprising a plurality of sequencing reads, for a DNA-containing sample of a subject, wherein the sequence data may be obtained by sequencing by synthesis; mapping the sequencing reads to a genome; identifying deletions in high-complexity sequence context; determining a deletion signal for the DNA-containing sample, wherein the deletion signal may comprise a distribution of non-clonal or subclonal deletions and microhomology patterns of DNA sequences flanking sites of mapped deletions in the genome of the subject; decomposing the deletion signal into classes such that deletions due to imperfect DNA repair can be separated from deletions resulting from systematic effects such as presence of personal deletion variants and false positive deletions arising from sample preparation, sequencing, and analysis; and quantifying the deletions resulting from imperfect DNA repair with mixture modeling to produce a quantified deletion distribution.

In some embodiments, the method may further comprise determining, based on the quantified deletion distribution, a clonal profile for the subject, wherein the clonal profile comprises at least one clonal deletion.

In some embodiments, the method may further comprise determining, based on the quantified deletion distribution, a subclonal profile for the subject, wherein the clonal profile comprises at least one subclonal deletion distinct from one or more clonal deletions.

In some embodiments, the method may further comprise determining a correlation between the quantified deletion distribution and one or more clonal substitutions.

In some embodiment, the correlation between the quantified deletion distribution and the one or more clonal substitutions comprises a correlation between the deletion distribution of the at least one subclonal deletion distinct from one or more clonal deletions and one or more patterns of the one or more clonal substitutions.

In some embodiments, the decomposing herein may comprise using sequence entropy to select high-complexity regions and exponential modeling to filter out the systematic effects. In some embodiments, the decomposing herein may comprise determining one or more vector properties based on alignment to a reference genome, the one or more vector properties selected from the group consisting of a microsatellite index, surrounding sequence entropy, an indicator of the presence of a genome-wide repetitive element, distance from the read start and read end, and personal variant determination.

In some embodiments, the personal variant determination vector property herein may be determined based on mapping the regions surrounding the putative deletions on all other reads in order to determine whether or not it is a personal variant that mappers failed to recognize in other reads.

In some embodiments, the decomposing herein may further comprise generating, based on the one or more vector properties, a receiver-operator characteristic (ROC) curve using exponential modeling. In some embodiments, tensorial blind source decomposition herein may be used to optimize the weights of the receiver-operator characteristics on the ROC curve to achieve optimal isolation of deletions. In some embodiments, methods herein may further comprise determining a ROC curve cutoff for isolating deletions using standard maximum likelihood reasoning

In some embodiments, the decomposing herein may comprise classifying the distributed deletions in the deletion signal based on deletion sequence length and adjacent microhomology pattern. In some embodiments, the DNA-containing sample may comprise a blood or tissue sample.

In some embodiments, methods herein may further comprise obtaining a whole genome sequencing (WGS) data set for the DNA-containing sample of the subject.

In some embodiments, methods herein may further comprise determining, based on the quantified deletion distribution, a mutational signature or biomarker corresponding to one or more cancers. In some embodiments, methods herein may further comprise modifying or formulating a cancer treatment for the subject based on the quantified deletion distribution or the mutational signature. In some embodiments, the one or more cancers may be a BRCA1 and/or BRCA2 mutation-positive cancer.

In some embodiments, methods herein may comprise assessing, based on the quantified deletion distribution, the significance of the variants of unknown significance (VUS) in the subject.

In some embodiments, methods herein may comprise a method of assessing and quantifying imperfect dsDNA break repair. In some embodiments, methods herein may comprise a method of diagnosing cancer. In some embodiments, methods herein may comprise a method for assessing the genotoxicity of a therapeutic treatment. In some embodiments, methods herein may comprise a method for assessing the genotoxicity of a therapeutic cancer treatment. In some embodiments, methods herein may comprise a method for the monitoring of cancer progression in a subject. In some embodiments, methods herein may comprise a method for the early detection of cancer. In some embodiments, methods herein may comprise a method for the prevention or treatment of cancer.

In some embodiments, methods herein may comprise a method for the personalization of treatment of cancer in a subject, the method comprising: determining whether cancer cells in the subject will be sensitive to the administration of a predetermined small molecule. In some embodiments, the predetermined small molecule may be a poly adenosine diphosphate (ADP) ribose polymerase (PARD) inhibitor. In some embodiments, the cancer herein may be a cancer with defects in BRCA1/2 genes.

Aspects of the present disclosure provide devices to perform methods herein. In some embodiments, devices herein may comprise: at least one processor coupled with a non-transitory computer-readable storage medium having stored therein instructions which, when executed by the at least one processor, causes the at least one processor to perform the methods herein, or any elemental step thereof.

### BRIEF DESCRIPTION OF THE FIGURES

The following drawings form part of the present specification and are included to further demonstrate certain aspects of the present disclosure, which can be better understood by reference to the drawing in combination with the detailed description of specific embodiments presented herein.

FIG. 1 is a schematic depicting a process used herein for mining data obtained from sequencing of a sample for detection of one or more genomic deletions in the sample and/or to determine a deletion signal for the sample.

FIG. 2 depicts a graph illustrating the properties of deletion signals for a cancer sample and a normal sample from a single donor. On x-axis the length of microhomologies at sites flanking deletions is displayed, on y-axis the number of subclonal deletions remaining after all filtering procedures is displayed.

FIGS. 3A-3D depict graphs of showing no deletion signals or very weak deletion signals for 4 representative donor samples. WGS data sets were obtained from the ICGC database.

FIGS. 4A-4D depict graphs showing a deletion signals obtained for 4 representative donors. The WGS data sets for analyzed samples were obtained from the ICGC database.

FIGS. 5A-5D depict graphs showing unexpected deletion signals for 4 representative donors. The WGS data sets for the analyzed samples were obtained from the ICGC database.

FIG. 6 depicts a graph showing no correlation of age with the magnitude of deletion signals for donor samples from the ICGC database.

FIG. 7 depicts the partitioning of cancer patients based on correlation between clonal deletion signal (y-axis, log10 scale) and subclonal deletion signal (x-axis, magnitude of deletion signal scale). The orange color indicates patients in which the magnitude of the subclonal deletion signal exceeded 20% of enrichment over background, while the blue color indicates patients for which the subclonal deletion signal have not reached that threshold.

FIGS. 8A-8D depict graphs of deletion signals calculated from sequencing read 2 (R2) or HCC1395BL (human control) and HCC1395 (human breast cancer) cells lines. WGS data sets used for this analysis were obtained from either of two different Illumina technologies (HiSeq2500 or HiSeq4000) using sequencing libraries prepared by two different approaches (Nextera or Kapa).

FIGS. 9A-9D depict graphs of microhomologies from sequencing read 1 (R1) for HCC1395BL (human control) and HCC1395 (human breast cancer) cells lines. WGS data sets used for this analysis were obtained from either of two different Illumina instruments (HiSeq2500 or HiSeq4000) using sequencing libraries prepared by two different approaches (Nextera or Kapa).

### DETAILED DESCRIPTION OF THE INVENTION

The defects in HRR are compensated for by other DNA repair pathways. These pathways may cause an elevated level of deletions which happen in random genomic locations and are different for each cell. For these deletions, DNA sequencing on a population of cells will result in deletion-containing sequencing reads that will map to a distinct position in the reference genome only once. It cannot be expected to observe those deletions multiple times before clonal expansion nor can it be expected to observe those deletions if they originate after several cell divisions involved in clonal expansion. These deletions and their properties are currently not examined despite its potential in diagnosis and treatment.

The disclosed methods analyze the deletion signal represented by the cumulative number of subclonal deletions, quantify the deletion signals patterns, and their results may be used to aid in the screening, the clinical diagnosis and treatment of diseases and/or conditions. Accordingly, the present disclosure generally relates to methods of collecting a sample from a subject, subjecting the sample to whole genome sequencing, detecting one or more genomic deletions in the results of sequencing by performing data mining on the sample's WGS data. The methods may, for example, aid in the in the screening, the clinical diagnosis and treatment of cancers. For example, methods of determining the deletion signal herein may allow for determination and administration of one or more cancer treatment regimens suitable for the subject. Further, methods herein can be used to determine the clonal and subclonal profiles of a cancer, which can be of prognostic value when treating the cancer.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

“Small molecules” as used herein can refer to chemicals, compounds, drugs, and the like.

The term “nucleic acid” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

It should also be understood that, unless clearly indicated to the contrary, in any methods claimed herein that include more than one step or act, the order of the steps or acts of the method is not necessarily limited to the order in which the steps or acts of the method are recited.

### I. Methods

In general, methods disclosed herein may be useful for the detection of one or non-clonal and/or subclonal deletions, especially those associated and/or those correlated (singly or in the aggregate) with various diseases, disorders and conditions including cancer. The methods disclosed herein may also be useful for identifying and selecting one or more therapies (e.g., cancer therapy) based on the one or more deletions detected. The HRR pathway is responsible for high-fidelity DNA double strand break (DSB) repair and involves numerous genes. Two example genes, include, but are not limited to, BRCA1 and BRCA2. Defects in HRR may be compensated for by other error-prone DNA repair pathways that often introduce short genomic deletions near sites of repair.

Briefly, the method may include determining a deletion signal for a DNA-containing sample of a subject, wherein the deletion signal comprises distributions (frequencies) of deletions with microhomologies of different lengths at the deletion sites in a DNA sequence or genome of the subject or sample thereof. The method may further include decomposing the deletion signal into components corresponding to changes arising from: (1) DNA repair processes, (2) systematic effects due to mapping personal deletion variants to reference genomes, and (3) false positive deletions generated during sample preparation, sequencing, and analysis, and quantifying these components to produce mutational signatures of defective HRR.

Methods herein can detect patterns consisting of frequencies of microhomologies having a length from “0” to whatever-is-the-longest microhomology detectable. In some aspects, each deletion detected using the methods herein may be a single special deletion (i.e., there are no other deletions like one non-clonal deletion). In some aspects, a single special deletion may be determined by mapping to a reference (e.g., a known genomic sequences, a plurality of known genomic sequences). In some aspects, after mapping, the sequence and two sites before and after a single special deletion can be determined. In some aspects, after the sequence and two sites before and after a single special deletion is determined, both ends may be examined to observe for microhomology, wherein the microhomology may have a length of 0 bp or more, 0 bp to about 50 bp, 0 bp to about 40 bp, 0 bp to about 30 bp, 0 bp to about 20 bp, or 0 bp to about 10 bp. In some aspects, methods herein may determine that a single special deletion can be designated as a number (e.g., “1 deletion”, “2 deletion”, and so forth) wherein the microhomology length of the single special deletion can be designated as a property of the numbered single special deletion (e.g., “microhomology length 10, 1 deletion”, “microhomology length 9, 2 deletion”, and so forth). In some aspects, methods herein may designated each single special deletion identified by the methods herein with a number and a property until all single special deletion have been designated. In some aspects, the designated single special deletions determined herein can be plotted as the number of subclonal deletion with a specific microhomology length (so histogram of subclonal deletions with microhomology lengths 0 to whatever was the longest).

### (a) Subjects and Samples

In some embodiments, the present disclosure provides methods of detecting one or more non-clonal and subclonal genomic deletions in a sample collected from a subject. As used herein, a suitable subject includes a mammal, a human, a livestock animal, a companion animal, a lab animal, or a zoological animal. In some embodiments, a subject may be a rodent, e.g., a mouse, a rat, a guinea pig, etc. In other embodiments, a subject may be a livestock animal. Non-limiting examples of suitable livestock animals may include pigs, cows, horses, goats, sheep, llamas and alpacas. In yet other embodiments, a subject may be a companion animal. Non-limiting examples of companion animals may include pets such as dogs, cats, rabbits, and birds. In yet other embodiments, a subject may be a zoological animal. As used herein, a “zoological animal” refers to an animal that may be found in a zoo. Such animals may include non-human primates, large cats, wolves, and bears. In other embodiments, the animal is a laboratory animal. Non-limiting examples of a laboratory animal may include rodents, canines, felines, and non-human primates. In some embodiments, the animal is a rodent. Non-limiting examples of rodents may include mice, rats, guinea pigs, etc. In preferred embodiments, the subject is a human.

In some embodiments, methods of detecting one or more non-clonal and subclonal genomic deletions in a sample collected from a subject herein may include subjecting at least one sample obtained from the subject to whole genome sequencing. In some embodiments, at least one sample can be obtained from a subject who has not been diagnosed with a disease and/or a condition. In some embodiments, at least one sample can be obtained from a subject who has been diagnosed with or is suspected of having a disease and/or a condition. In some embodiments, the disease and/or condition is cancer. In some embodiments, at least one sample can be obtained from a subject who has not been diagnosed with a cancer. In some embodiments, at least one sample can be obtained from a subject suspected of having cancer. In some embodiments, at least one sample can be obtained from a subject who has been diagnosed with a cancer. In some embodiments, at least one sample can be obtained from a subject who has been diagnosed with a cancer suspected of having deficient HRR. In some embodiments, at least one sample can be obtained from a subject who has been diagnosed with a cancer suspected of having one or more non-clonal or subclonal genomic deletions.

In some embodiments, at least one sample can be obtained from a subject who presents with at least one symptom of a cancer suspected of having deficient HRR. In some embodiments, at least one sample can be obtained from a subject who presents with at least one symptom of a cancer suspected of having one or more genomic deletions. In some embodiments, at least one sample can be obtained from a subject who presents with at least one symptom of a cancer suspected of having one or more non-clonal or subclonal genomic deletions. Non-limiting symptoms of a cancer suspected of having deficient HRR, having one or more genomic deletions, and/or having one or more subclonal genomic deletions, include the cancer exhibiting platinum sensitivity, PARP-inhibitor sensitivity, or a combination thereof. In some embodiments, at least one sample can be obtained from a subject who has been diagnosed with a cancer and the cancer has demonstrated a prior platinum sensitivity. In some embodiments, at least one sample can be obtained from a subject who has been diagnosed with a cancer and the cancer has demonstrated a prior sensitivity to PARP inhibitors.

In some embodiments, at least one sample can be obtained from a subject who has been diagnosed with a cancer and the cancer has been classified into one of the five stages of cancer. The method of staging a cancer stage can include assessing the size of the tumor, which parts of the organ have cancer, whether the cancer has spread (metastasized), where it has spread, and the like. One of skill in the art will appreciate that one or more staging systems can be used depending on the cancer type. In some embodiments, at least one sample can be obtained from a subject who has been diagnosed with a cancer classified into one of the five stages of cancer according to the TNM system. In the TNM system: T stands for tumor. It describes the size of the main (primary) tumor. It also describes if the tumor has grown into other parts of the organ with cancer or tissues around the organ. T is usually given as a number from 1 to 4. A higher number means that the tumor is larger. It may also mean that the tumor has grown deeper into the organ or into nearby tissues. N stands for lymph nodes. It describes whether cancer has spread to lymph nodes around the organ. NO means the cancer hasn't spread to any nearby lymph nodes. N1, N2 or N3 means cancer has spread to lymph nodes. N1 to N3 can also describe the number of lymph nodes that contain cancer as well as their size and location. M stands for metastasis. It describes whether the cancer has spread to other parts of the body through the blood or lymphatic system. M0 means that cancer has not spread to other parts of the body. M1 means that it has spread to other parts of the body. In some aspects, the TNM description can be used to assign an overall stage from 0 to 4 for many types of cancer. Stages 0 to 4 are can present as described in Table 1.

TABLE 1

| Stage | Features |
|---|---|
| Stage 0 | Abnormal cells are present but have not spread to nearby tissue. |
| | Also called carcinoma in situ. |
| Stage 1 | the tumor is usually small and hasn't grown outside of the |
| | organ of origin |
| Stage 2 | the tumor is larger but has not grown outside of origin |
| Stage 3 | the tumor is larger and has grown outside of the organ of origin |
| | in to nearby tissue |
| Stage 4 | the cancer has spread through the blood or lymphatic system to a |
| | distant site in the body (metastatic spread) |

In some embodiments, at least one sample can be obtained from a subject who has been diagnosed with a stage 0, stage 1, stage 2, stage 3, or stage 4 cancer. In some embodiments, at least one sample can be obtained from a subject who has been diagnosed with a stage 0, stage 1, stage 2, stage 3, or stage 4 cancer wherein the cancer can be breast, ovarian, prostate, melanoma, lung or pancreatic cancer. In some embodiments, at least one sample can be obtained from a subject who has been diagnosed with a stage 3 or stage 4 cancer. In some other examples, at least one sample can be obtained from a subject who has been diagnosed with a stage 3 or stage 4 cancer, wherein the cancer can be, but is not limited to, breast, ovarian, prostate, melanoma, lung or pancreatic cancer.

In some embodiments, at least one sample can be obtained from a subject who has at least one solid tumor. In some embodiments, at least one sample can be obtained from a subject who has at least one solid tumor that meets the criteria of stage 1, stage 2, stage 3, or stage 4 cancer. In some embodiments, at least one sample can be obtained from a subject who has at least one solid tumor that meets the criteria of stage 1, stage 2, stage 3, or stage 4 cancer wherein the solid tumor can be a breast, ovarian, prostate, melanoma, lung or pancreatic tumor. In some embodiments, at least one sample can be obtained from a subject who has at least one solid tumor that meets the criteria of stage 3 or stage 4 cancer. In some embodiments, at least one sample can be obtained from a subject who has at least one solid tumor that meets the criteria of stage 3 or stage 4 cancer wherein the solid tumor can be a breast, ovarian, prostate, melanoma, lung or pancreatic tumor.

In some embodiments, a sample obtained from a subject to be used in any of the methods disclosed herein may be a tissue sample, a blood sample, a plasma sample, a lavage, a cell, a stool sample, a hair sample, venous tissues, cartilage, a sperm sample, a skin sample, an amniotic fluid sample, a buccal sample, saliva, urine, serum, sputum, bone marrow or a combination thereof. In some embodiments, a sample obtained from a subject to be used in any of the methods disclosed herein may be a tumor sample. Non-limiting methods suitable for use herein to collect tumor samples include collection fine needle aspirate, removal of pleural or peritoneal fluid, excisional biopsy, and the like. In some embodiments, a tumor sample can include a biopsy from a single tumor, a biopsy from at least one tissue in contact with the tumor, and any combination thereof. In some embodiments, a biopsy sample of the tumor and/or at least one tissue in contact with the tumor can be from about 1 mg about 50 mg (e.g., about 1 mg, 2 mg, 4 mg, 6 mg, 8 mg, 10 mg, 15 mg, 20 mg, 25 mg, 30 mg, 35 mg, 40 mg, 45 mg, 50 mg) of tissue per sample.

In some embodiments, a sample obtained from a subject to be used in any of the methods disclosed herein may be a blood and/or plasma sample. In some embodiments, genetic material originating from a tumor cell may be isolated from the blood or plasma sample from the subject, as tumor DNA may be shed into the bloodstream. In some embodiments, a tumor sample for use in the methods herein can be tumor DNA isolated from a blood sample collected from any of the subjects disclosed herein.

### (b) Genome Sequencing

In some embodiments, methods of detecting one or more genomic deletions in a sample collected from a subject herein may comprise subjecting the sample to whole genome sequencing. In some embodiments, methods of determining a deletion signal, wherein a deletion signal comprises a cumulative number of distributed non-clonal and subclonal deletions in a sample collected from a subject herein may comprise subjecting the sample to whole genome sequencing. In some embodiments, methods of determining a clonal profile, a subclonal profile, or both in a sample collected from a subject herein may comprise subjecting the sample to whole genome sequencing.

Any suitable technique for sequencing genetic material from the one or more samples disclosed herein can be used in various embodiments of the present methods. In some embodiments, sequencing genetic material from the one or more samples disclosed herein may be performed using next-generation sequencing (NGS) technologies. Apparatuses and materials for carrying out such sequencing techniques are well-known in the art and are commercially available. Non-limiting examples of apparatuses suitable for use herein can include Illumina systems (e.g., HiSeq 1000 System; HiSeq 1500 System; HiSeq 2000 System; HiSeq 2500 System; HiSeq 3000 System; HiSeq 4000 System; HiSeq X Five System; HiSeq X Ten System; NextSeq 1000 System; NextSeq 2000 System; NextSeq 500 System; NextSeq 550 System; NovaSeq 6000 System), MGI systems (e.g., DNBSEQ-T7; DNBSEQ-G400), Singular Genomics systems (e.g., G4), Sequencing By Synthesis (SBS), Sequencing By Binding (SBB), and the like.

In some embodiments, DNA sequencing libraries generated for sequencing methods herein may be constructed using methods known in the art. Non-limiting examples include ligation-based library construction, tagmentation (e.g., use of a transposase enzyme to simultaneously fragment and tag DNA in a single-tube reaction), and the like. In some embodiments, DNA sequencing libraries generated for sequencing methods herein may be constructed using commercially available library preparation kits (e.g. Nextera XT DNA Library Preparation Kit, Illumina® DNA PCR-Free Prep, Illumina® DNA Prep, KAPA HyperPlus Kit PCR-free and with PCR amplification, KAPA HyperPrep Kit PCR-free and with PCR amplification, MGIEasy Universal DNA Library Prep).

In some embodiments, DNA sequencing libraries generated for sequencing methods herein are first screened for one or more damaged bases before sample DNA is sequenced. Abasic sites are a family of DNA lesions that lack the heterocycles involved in Watson-Crick base pair formation in duplex DNA. Abasic sites may be present in the sample and they may generate deletions and indels in the results of sequencing reactions. The type of deletion and the type of inserted base may depend on the polymerase used in sequencing reactions. In some embodiments, a mix of randomized oligonucleotides with damaged bases may be added during sequencing as internal controls to obtain patterns of deletions generated by specific polymerases used in a particular library preparation and sequencing reactions. In some embodiments, expected patterns may be included in the data model during computations detailed herein.

In some embodiments, samples disclosed herein may be subjected to low-pass sequencing using short-read sequencing. As used herein, short-read sequencing can read up to about 150 base pair (bp) to about 800 bp per a sequencing read. In some embodiments, samples disclosed herein can be subjected to low-pass sequencing using long-read sequencing. As used herein, long-read sequencing can read at least about 10 kilobases (kb) per read. Commercial platforms suitable for use long-read sequencing herein can include, but are not limited to, those developed by Pacific Biosciences.

### (c) Data Mining and Analysis of Sequence Data

In some embodiments, sequencing data obtained according to the methods disclosed herein may be subjected to data mining. The presently disclosed methods are capable of analyzing the signals represented by the distributions of non-clonal deletions together with the properties of these distributions. In some embodiments, the deletions may be classified and quantified using data mining methods that categorizes the sites of detected deletions based on their length, the patterns of sequence complementarity surrounding deletion sites, and/or other features. In some embodiments, categorizing the sites of detected deletions according to the methods herein allow for deletions originating from imperfect DNA repair to be differentiated from deletions representing personal variants and deletions due to false positives arising from DNA damage introduced during sample handling, genetic material isolation, sequencing library preparation, sequencing process, and sequencing data analysis.

In some embodiments, methods herein may include providing sequence data for a DNA-containing sample of a subject. In some embodiments, the sequence data may include a plurality of sequencing reads and be obtained by sequencing by synthesis. In some embodiments, sequence data to be subjected to data mining methods disclosed herein may be data for the entire genome. In some embodiments, sequence data to be subjected to data mining methods disclosed herein may be one or more segments of the entire genome. In some embodiments, sequence data to be subjected to data mining methods disclosed herein may be one or more segments of the entire genome having repetitive sequences. Repetitive DNAs can include both short and long sequences that repeat in tandem or are interspersed throughout the genome, such as transposable elements (TE), ribosomal rRNA genes (rDNA), and satellite DNA. In some embodiments, sequence data to be subjected to data mining methods disclosed herein may be one or more types of repetitive sequences, including but not limited to centromere sequences, mitochondrial sequences, and the like.

In some embodiments, methods herein may further include mapping the sequencing reads to a genome and identifying deletions in high-complexity sequence context. In some embodiments, methods herein may further include determining a deletion signal for the DNA-containing sample, wherein the deletion signal comprises a distribution of non-clonal or subclonal deletions and microhomology patterns of DNA sequences flanking sites of mapped deletions in the genome of the subject or tissue sample thereof. In some embodiments, methods herein may further include decomposing the deletion signal into classes such that deletions due to imperfect DNA repair can be separated from deletions resulting from systematic effects such as presence of personal deletion variants and false positive deletions arising from sample preparation, sequencing, and analysis. In some embodiments, methods herein may include quantifying the deletions resulting from imperfect DNA repair with mixture modeling to produce a quantified deletion distribution. False positive deletions due to sequencing process and sequencing data analysis may result from: (1) incorrect mapping of sequencing reads to the genome; (2) polymerase slippage during PCR or polony amplification; (3) mispriming during PCR or polony amplification; and (4) hybrids formed during PCR or polony amplification. These four mechanisms have specific properties that allow for their identification and isolation from the signal. In some embodiments, methods herein may use entropy and/or mixture modeling to filter out these systematic effects. In some embodiments, methods herein may use one or more of the following to avoid false positive deletions: avoiding damage during sample handling from retrieval to sequencing library preparation, using enzymes that cleave DNA at abasic sites, using Nextera (or a similar sequencing library preparation method) to reduce mispriming.

In some embodiments, sequence data obtained according to the methods disclosed herein can be are aligned to a reference genetic material, for example to one or more reference genomes. In some embodiments, one or more reference genomes can be a genome corresponding to the organism of the subject from which the genetic sample was obtained (e.g., a human reference genome if the subject is human), or these can be reference genomes corresponding to organisms which are different from the individual from which the genetic sample was obtained. In some embodiments, one or more reference genomes may be a pangenome. Example human reference genomes suitable for use herein may include one or more publicly available human reference genomes. Non-limiting examples of publicly available human reference genomes include the hg19 human reference genome (Kent et al., Genome Res. 2002 June; 12(6):996-1006)) and phases 1-3 of the International Genome Sample Resource (www.internationalgenome.org).

In some embodiments, sequence data obtained according to the methods disclosed herein may be aligned to a reference genome using software (i.e., “aligners”) that may implement an algorithm. In some aspects, suitable publicly- or commercially-available aligners for aligning sequencing reads herein to reference genomes according to the present methods are well-known to those of ordinary skill in the art, and include, for example but not limited to BWA or Bowtie 2.

In some embodiments, sequence data obtained according to the methods disclosed herein may be aligned to a reference genome, then one or more identified deletions may be recovered. In some embodiments, one or more identified deletions may be recovered and each of them may be characterized by a vector property. In some embodiments, the decomposing of the deletion signal may include using sequence entropy to select high-complexity regions and exponential modeling to filter out the systematic effects that mimic deletion signals. In some embodiments, the decomposing of the deletion signal may comprise determining one or more vector properties based on alignment to a reference genome. In some embodiments, a vector property may include microsatellite index, entropy of sequences surrounding the mapped deletion, indicator of the presence of a genome-wide repetitive element, distance from the read start and read end, or any combination thereof. In some embodiments, an additional vector property may arise from mapping the regions surrounding the putative deletions on all other reads in order to determine whether or not it is a personal variant that mappers failed to recognize in other reads.

In some embodiments, sequence data obtained according to the methods disclosed herein may be subjected to exponential modeling. In some embodiments, exponential modeling based upon a vector property may define the receiver-operator characteristic (ROC) curve, while tensorial blind source decomposition may optimize the weights of these characteristics to achieve the best separation of different types of deletions, as described by the ROC curve. In some embodiments, the ROC curve cutoff for differentiating between artifacts and legitimate deletions is determined by standard maximum likelihood reasoning. In some embodiments, the decomposing of the deletion signal may include classifying the distributed deletions in the deletion signal based on deletion sequence length and adjacent microhomology patterns.

In some embodiments, methods of analyzing sequence data herein can include quantifying mutations in sequencing. In conventional approaches to quantifying mutations in sequencing, a sequence variant present only once in a pool of all sequencing reads is ignored and, in most applications, this also applies to a variant observed two or three times across all sequencing reads. This limits all mutation studies to clonally amplified variants where the clonal amplification happened in a tissue, e.g., during cancer growth, or was introduced by PCR or multiple displacement amplification (MDA) during sequencing library preparation. In some embodiments, extraordinarily rare, non-recurring-in-data events may be counted after separating non-recurring events resulting from biologically relevant processes from those arising from sequencing errors, artifacts of data analysis, replication errors, personal variants, or a combination thereof. In some embodiments, extraordinarily rare, non-recurring-in-data events may be counted as real signal by associating with each potential source of deletion and/or deletion-like signals functions, describing expectation regarding observing source-specific patterns in sequencing data, training these functions on whole genome sequencing data from variable sources, recovering the source-specific patterns, and validating that these patterns do not show characteristic correlations indicating a systematic effect that needs to be included in the data analysis.

In some embodiments, sequence data obtained according to the methods herein may be aligned to a reference genome according to methods described herein, resulting in a “mapped read” (also referred to herein as “mapped read data.”) In some embodiments, mapped read data may be subjected to data mining to identify one or more genomic deletions. In some embodiments, mapped read data may be subjected to data mining comprised of one or more sequential methods of data filtering to identify one or more genomic deletions. In some embodiments, mapped read data may be subjected to data mining comprised of multiple filters to identify one or more genomic deletions.

In some embodiments, mapped read data can be filtered for removal of biological and/or technical background artifacts. Biological background is mostly slippage errors during replication. Technical background includes slippage errors, hybridization artifacts, and incorrect/inconsistent mapping of reads. In some embodiments, mapped read data can be filtered for removal of tandem repeats, deletions of less than about 10 base pairs (bp) (e.g., about 1, 2, 3, 4, 5, 6, 7, 8, 9, 10 bp), approximate tandem repeats, locally repetitive sequences, optional rejection of globally repetitive sequences, deletions too close to read ends, read pairs that are discordantly mapped, reads with too many substitution errors, deletions observed elsewhere, or any combination thereof.

In some embodiments, mapped read data can be filtered for removal of paired-end sequencing reads that map more than at least 1000 bp apart. In some embodiments, mapped read data can be filtered for removal of mapped read with poor mapping quality score (MAPQ). A mapping quality score describes the probability that a sequencing read is aligned incorrectly. In some embodiments, mapped read data can be filtered based on invalid TLEN (signed observed Template LENgth) values. Two paired end sequencing reads result from the same sequencing polony so they both should be measured, and they should map to the same chromosome and within reasonable distance from each other. Mapping of only one read out of two paired end sequencing reads to reference genome indicates problems with the polony so it's better to filter out such reads. In some embodiments, mapped read data can be filtered for removal of hard clipped reads. In hard clipped reads, part of the sequence has been removed prior to alignment due to problems with sequencing quality. Even if parts of such reads may map well, the quality problem might be leaking out to other parts of the reads and may contaminate the analysis. In some embodiments, mapped read data can be filtered for removal of mapped reads in which paired end sequencing reads map to different chromosomes. In some embodiments, mapped read data can be filtered in for removal of mapped reads with unidirectional mapping. In some embodiments, mapped read data can be filtered for removal of mapped reads without deletions.

In some embodiments, mapped read data can be filtered for removal of population polymorphisms. In some aspects, mapped read data can be filtered using known data on population sequence polymorphisms i.e. sequence variants present in human populations. The curated from publicly available datasets such as, but not limited to, the dbSNP152 and gnomAD databases. In some aspects, mapped read data can be filtered for personal polymorphism using WGS data for a particular sample or group of samples. In some embodiments, mapped read data can be filtered for removal of repetitive sequences or reads mapping to repetitive regions reads. In some embodiments, mapped read data can be filtered for removal of sequencing reads with the excessive number of errors.

In some embodiments, mapped read data can be filtered for removal of hybrids. In some embodiments, mapped read data can be filtered for removal of deletions shorter than about 10 bp (e.g., about 1, 2, 3, 4, 5, 6, 7, 8, 9, 10 bp). In some embodiments, mapped read data can be filtered in for removal of mapped reads containing low complexity sequences. Reads with low complexity sequences may contain stretches of homopolymer nucleotides or simple sequence repeats.

In some embodiments, mapped read data may be subjected to data mining to identify one or more genomic deletions with sequence microhomology at deletions' flanking sites. Short regions of DNA sequence homology, called ‘microhomology’ can occur at certain germline and somatic breakpoint junctions. Microhomology herein refers to the repeat of a sequence at the start of the deletion and just after the deletion, with the repeated region being relatively short. Although definitions of breakpoint microhomology vary with respect to the length of the homologous region, it can be defined as a series of nucleotides that are identical at the junctions of the two genomic segments that contribute to the rearrangement. Microhomology has also been reported in DNA sequences that are adjacent to, but do not overlap, breakpoint junctions. Appearance of deletions not in tandem repeats but that have short microhomology is characteristic of specific defects in DNA repair. In some embodiments, mapped read data may be subjected to data mining to identify one or more genomic deletions with microhomology lengths of less than about 10 (e.g., about 10, 9, 8, 7, 6, 5, 4, 3, 2, 1) at sequences near the deletion site. In some embodiments, mapped read data may be subjected to data mining to identify one or more genomic deletions having microhomology at sequences near the deletion site, which are related to mutations in BRCA1 and/or BRCA2 genes.

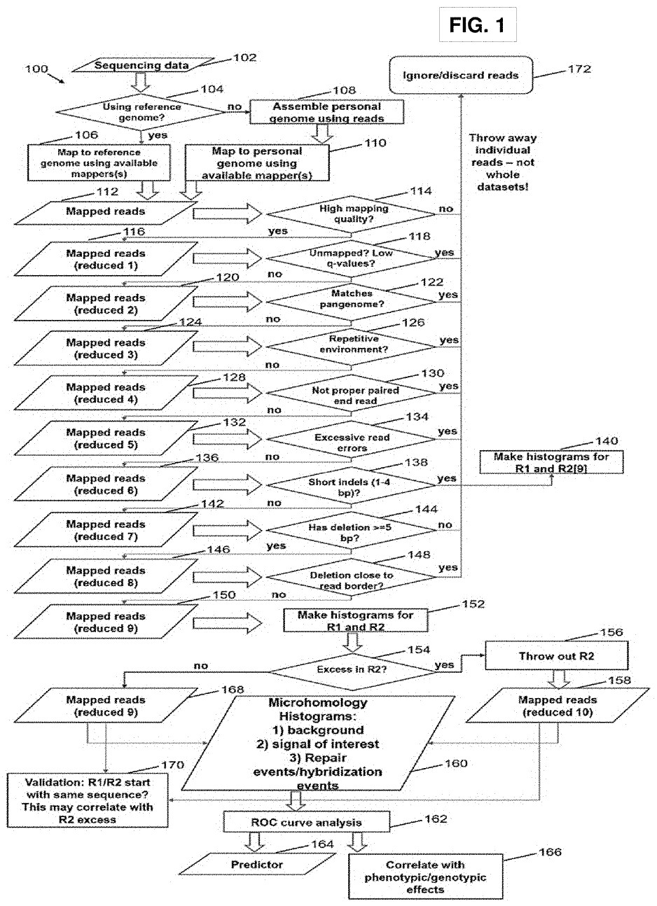

In an exemplary embodiment, data mining according to the methods disclosed herein may follow any of the steps provided in FIG. 1.

### (d) Clonal and Subclonal Profiles

Cancer cells gain the ability grow in an unchecked manner by acquiring driver mutations. Some cancers have mutations that result in mutator phenotypes (i.e. the mutation rate of cancer tissue is higher than of normal tissue) and some mutators can be drivers of cancers. Cancers with driver mutations undergo fast clonal expansion that makes acquisition of subsequent passenger and driver mutations more likely. Depending on time of introducing a mutation during tumor growth it may be uniformly present in the tumor or it may be present only sporadically. Such subclonal mutations, which are passed on only to the subpopulation of cells in the tumor. Cancer cells in each subclone have the founding mutations and the subclonal mutations. The result of the accumulation of clonal and subclonal mutations is a tumor that is composed of a heterogeneous mixture of cells.

In some embodiments, the methods disclosed herein may be used to determine a clonal profile, a subclonal profile, or both of a subject herein. In some embodiments, methods herein may detect and classify non-clonal or low clonality deletions that map to high-complexity regions of the genome using sequencing with synchronized and amplified readout.

In some embodiments, a clonal profile may be generated using the methods herein. A deletion signal may be determined using the methods herein from samples collected from one or more subjects having a disease and/or condition (e.g., a cancer) to establish a catalogue of deletion signals (i.e., a clonal profile) frequently associated with that disease and/or condition. In some embodiments, a quantified deletion distribution determined by the methods herein may be used to generate at least one clonal profile for a subject herein, wherein the at least one clonal profile may comprise at least one clonal deletion. In some embodiments, the at least one clonal profile includes 10 or more deletions, 50 or more deletions, 100 or more deletions, 200 or more deletions, 500 or more deletions, 1,000 or more deletions, 5,000 or more deletions, or 10,000 or more deletions.

In some embodiments, one or more deletion signals determined using the methods herein that are frequently associated with a disease and/or condition (e.g., a cancer) may be removed from the clonal profile generated herein of that disease and/or condition to detect one or more deletion signals that are rarely associated with that disease and/or condition.

In some embodiments, the one or more deletion signals that are rarely associated with a disease and/or condition (e.g., a cancer) detected using the methods herein may establish a sub-clone profile for that disease and/or condition. In some embodiments, a quantified deletion distribution determined by the methods herein may be used to generate at least one subclonal profile for a subject herein, wherein the at least one subclonal profile may comprise at least one subclonal deletion that is distinct from one or more clonal deletions. In some embodiments, the at least one subclonal profile includes 10 or more deletions, 50 or more deletions, 100 or more deletions, 200 or more deletions, 500 or more deletions, 1,000 or more deletions, 5,000 or more deletions, or 10,000 or more deletions.

In some embodiments, methods herein may be used to determine one or more correlations between subclonal deletion distributions and the number of clonal substitutions. Wherein a “deletion” occurs when one or more nucleic acid bases are deleted from the genomic sequence, a “substitution” occurs when one or more nucleic acid bases in the genomic sequence is replaced by the same number of bases (for example, an endogenous cytosine substituted for an adenine). In some embodiments, a quantified deletion distribution determined by the methods herein may be used to generate at least one subclonal profile wherein the subclonal deletion distributions correlate to the number of clonal substitutions, the type of clonal substitutions (i.e., patterns), or both. In some embodiments, a quantified deletion distribution determined by the methods herein may be used to generate at least one subclonal profile wherein the subclonal deletion distributions may predict the number of clonal substitutions, the type of clonal substitutions (i.e., patterns), or both. In some embodiments, a quantified deletion distribution determined by the methods herein may be used to generate at least one subclonal profile wherein the subclonal deletion distributions may predict the number of clonal substitutions, the type of clonal substitutions (i.e., patterns), or both and the predicted number of clonal substitutions and/or type of clonal substitutions can be used to diagnose a disease and/or a condition (e.g., cancer). In some embodiments, a quantified deletion distribution determined by the methods herein may be used to generate at least one subclonal profile wherein the subclonal deletion distributions may predict the number of clonal substitutions, the type of clonal substitutions (i.e., patterns), or both and the predicted number of clonal substitutions and/or type of clonal substitutions can be used to treat a disease and/or a condition (e.g., cancer).

In some embodiments, the deletion signal herein may be used in constructing a phylogenetic map of the clonal and subclonal populations. As used herein, a “phylogenetic map” or “phylogeny” as it relates to subclonal populations is an organization or clustering of various subclonal populations based on the patterns of mutations that reflect the evolution of cancer cells within a tumor or the drift in normal cells. In some embodiments, phylogenetic maps may be phylogenetic trees, which can be classified in different ways, such as by shape (linear vs. branching), number of subpopulations (e.g. monoclonal for a single population, polyclonal for >1), and/or number of ancestral tumors.

### II. Administration of Treatment

The presently disclosed methods and devices detect and quantify mutational signatures resulting from reduced effectiveness of HRR. All cancers and other conditions in which effectiveness of HRR is reduced should produce signatures that are detectable and quantifiable by the presently disclosed methods and devices. In some embodiments, methods and devices disclosed herein may be used in the early diagnosis and monitoring of cancers, including cancers in which BRCA1/2 are mutated, where defects in HRR contribute to the cancer onset and progression.

The presently disclosed methods and devices solve several problems by providing for: (1) early detection of cancers where HRR is defective; (2) assessment of the significance of variants of unknown significance (VUS); (3) personalization of cancer treatments by detecting whether a specific cancer will be sensitive to PARP inhibitors or other similar treatments; and (4) characterization of cancer growth from the start of the clonal expansion which may provide actionable information.

Additionally, the presently disclosed methods and devices offer the following advantages over conventional technologies by: (1) analyzing a unique signal that appears before the onset of cancer that is currently ignored despite its potential to become a biomarker; (2) determining the number of rare and distributed deletions which is a phenotypic readout that can be detected even if the genotype responsible for generating the signal is unknown, thereby providing a method to assess the significance of the variants of unknown significance (VUS) in HRR-related gene and also provides many opportunities to personalize treatments and assess their safety including testing whether current drugs or treatments have specific genotoxicity; and (3) implementing a unique computational approach that relies on standard sequencing data that does not require special sample preparation.

The presently disclosed methods and devices may analyze the phenotypic readout (i.e., presence of a higher than expected number of non-clonal and subclonal deletions with the associated sequence features of their genomic environment) so that cancers can be detected even if the genetic changes responsible for their development are unknown. HRR defects also appear later in cancer progression, for instance in some prostate cancers, and sensitize cancer cells to specific treatments. In these cancers, the presently disclosed method and devices can be used to guide the choice of treatments. Many genetic changes have uncertain consequences and one of the greatest challenges in the cancer field is the assessment of the phenotypic significance of mutations present in cancer-related genes. The presently disclosed methods and devices provide a phenotypic readout. Therefore, when the elevated level of mutations is detected, it may be used to determine the significance to variants of unknown significance (VUS).

The present disclosure provides methods for quantifying levels of non-clonal or subclonal deletions in whole genome sequencing (WGS) data obtained with sequencing by synthesis approaches and combined with new approaches of analyzing these data. Although deletion signals are not amplified and fixed yet by cancer growth, a sample from patients carrying HRR defects that may lead to cancer may show a higher number of non-clonal and subclonal deletions than the number of non-clonal and subclonal deletions in tissues of non-carriers.

Therefore, quantifying the levels of non-clonal and subclonal deletions may help to diagnose many types of cancer earlier, as well as to better characterize the evolution of the cancer in a subject. Additionally, inactivation of the dsDNA break repair pathways may sensitize the cells of a subject to various treatments including, for example, poly ADP ribose polymerase (PARP) inhibitors. According to at least one aspect of the present disclosure, the presently disclosed methods may provide a means to mitigate the resistance and oversensitivity to personalized cancer treatments and therapies. Additionally, the presently disclosed methods of diagnosis and cancer assessment may be used to guide clinical decisions and treatments. The number of deletions for a sample can be used in rational drug design and discovery. For example, the manner by which the administration of small molecules affects the accumulation of deletions may be monitored and/or the genotoxicity of various substances or treatments may be assessed. Additionally, variants of unknown significance (VUS), which for instance represent over 10% of all variants detected in BRCA1/2 genes, can be analyzed for an increased level of deletions which may provide a functional readout for the variant and allow for associating a significance to it.

In some embodiments, treatment of a subject after quantifying the levels of non-clonal and subclonal deletions according to the methods disclosed herein, may prevent cancer progression. In some embodiments, treatment of a subject after quantifying the levels of non-clonal and subclonal deletions according to the methods disclosed herein, may ameliorate one or more symptoms associated with cancer. In some embodiments, treatment of a subject after quantifying the levels of non-clonal and subclonal deletions according to the methods disclosed herein, may reduce risk of cancer recurrence in the subject In some embodiments, treatment of a subject after quantifying the levels of non-clonal and subclonal deletions according to the methods disclosed herein, may slow tumor growth in the subject. In some embodiments, treatment of a subject after quantifying the levels of non-clonal and subclonal deletions according to the methods disclosed herein, may reduce the risk of metastasis in the subject.

According to embodiments of the present disclosure, methods herein may detect and/or classify non-clonal or low clonality deletions that map to high-complexity regions of the genome using sequencing with synchronized and amplified readout. In some embodiments, methods herein may include, among other features, (a) detecting all deletions by mapping sequencing reads to the genome; (b) calculating various properties and associate them with deletions; (c) decompose the deletion signal based on these properties so that deletions are categorized (false positives, personal variants, etc.); (d) use mixture modeling on the remaining part; (e) count genuine deletions and deletions attributed to specific categories; (f) check whether the counts correspond to increased levels of deletions over baselines.

In some embodiments, a subject determined to have a deletion signal according to the methods disclosed herein can be administered one or more anticancer therapies. Anticancer therapy as used herein refers to a treatment regimen for the treatment of malignant, or cancerous disease. Non-limiting examples of anticancer therapies can include administration of an anticancer drug, radiation, surgical methods, and the like. As used herein an “anticancer drug” refers to any drug with an intended use for the treatment of malignant, or cancerous disease. Anticancer drugs can be classified into three groups: cytotoxic drugs, hormones, and signal transduction inhibitors. Cytotoxic anticancer drugs suitable for use herein can include, but are not limited to: alkylating agents (e.g., nitrogen mustards and nitrosoureas); antimetabolites (e.g., folate antagonists, purine and pyrimidine analogues); antibiotics and other natural products (e.g., anthracyclines and vinca alkaloids); antibodies that improve drug specificity, and other generally cytotoxic drugs. In some embodiments, anticancer drugs herein can refer to platinum-based chemotherapeutics. In some embodiments, anticancer drugs herein can refer to PARP inhibitors. PARP inhibitors are a group of pharmacological inhibitors of the enzyme poly ADP ribose polymerase (PARP). Non-liming examples of PARP inhibitors suitable for use herein includes Olaparib, Rucaparib, Niraparib, Talazoparib, Veliparib, Pamiparib (BGB-290), CEP 9722, E7016, 3-Aminobenzamide, and any combination or derivative thereof.

In some embodiments, a subject determined to have a deletion signal according to the methods disclosed herein can be administered one or more anticancer therapies to treat a solid tumor. In some embodiments, anticancer therapies to be administered in accordance with the deletion signal as determined herein can re-sensitize or sensitize a tumor in a subject to one or more anticancer drugs (e.g., platinum-based chemotherapies). In some embodiments, anticancer therapies to be administered in accordance with the deletion signal as determined herein can re-sensitize or sensitize a tumor in a subject to one or more anticancer drugs to reduce costs, improve outcome and reduce or eliminate patient exposure to an anticancer therapy without significant effect. In some embodiments, a subject can have an anticancer drug resistant cancer or be suspected of developing such a cancer where additional agents can be administered to re-sensitize or sensitize the cancer in a subject.

In some embodiments, a subject determined to have a deletion signal according to the methods disclosed herein can have an anticancer drug resistant tumor or be suspected of developing such a tumor where additional agents can be administered to re-sensitize or sensitize a tumor in a subject wherein the tumor can include a solid tumor. In some embodiments, a solid tumor can be an abnormal mass of tissue that is devoid of cysts or liquid regions within the tumor. In some embodiments, solid tumors can be benign (not progressed to a cancer), a malignant or metastatic tumor. In some embodiments, a solid tumor herein can be a malignant cancer that has metastasized. In other embodiments, solid tumors contemplated herein can include, but are not limited to, sarcomas, carcinomas, lymphomas, gliomas or a combination thereof. In accordance with some embodiments herein, tumors resistant to anticancer drugs (e.g., platinum-based chemotherapies) can include, but are not limited to, a testicular tumor, ovarian tumor, cervical tumor, a kidney tumor, bladder tumor, head-and-neck tumor, liver tumor, stomach tumor, lung tumor, endometrial tumor, esophageal tumor, breast tumor, cervical tumor, central nervous system tumor, germ cell tumor, prostate tumor, Hodgkin's lymphoma, non-Hodgkin's lymphoma, neuroblastoma, sarcoma, multiple myeloma, melanoma, mesothelioma, osteogenic sarcoma or a combination thereof. In some embodiments, a targeted tumor contemplated herein can include a solid tumor such as a breast tumor, ovarian tumor, prostate tumor, melanoma, lung tumor, pancreatic tumor or any combination thereof.

Some standards of care in the art for solid tumors can include combination therapies. In some embodiments, anticancer therapies to be administered in accordance with the deletion signal as determined herein can be combination of at least two anticancer drugs. In some embodiments, anticancer therapies to be administered in accordance with the deletion signal as determined herein can be combination of at least a chemotherapeutic and an anticancer drug. In some embodiments, anticancer therapies to be administered in accordance with the deletion signal as determined herein can be combination of at least one platinum-based chemotherapeutic and at least one PARP inhibitor.

As used herein, a “platinum-based chemotherapeutic” is a chemotherapeutic that is an organic compound which contains platinum as an integral part of the molecule. In some embodiments, compositions of use herein can contain one or more platinum-based chemotherapeutics including, but not limited to, cisplatin, carboplatin, nedaplatin, triplatin tetranitrate, phenanthriplatin, picoplatin, satraplatin or a combination thereof. In some embodiments, a platinum-based chemotherapeutic can be administered separately from the compounds disclosed herein. In some embodiments, compositions containing a platinum-based chemotherapeutic of use herein can contain a concentration of the platinum-based chemotherapeutic at about 1 mg/ml to about 100 mg/ml (e.g., about 1 mg/ml, about 5 mg/ml, about 10 mg/ml, about 20 mg/ml, about 30 mg/ml, about 40 mg/ml, about 50 mg/ml, about 60 mg/ml, about 80 mg/ml, about 100 mg/ml). In some embodiments, the platinum-based chemotherapeutic or salt thereof or derivative thereof includes cisplatin. In certain embodiments, platinum-based chemotherapeutic agents can be administered to a subject alone or in combination with at least one at least one anticancer drug (e.g. PARP inhibitor), daily, every other day, twice weekly, every other day, every other week, weekly or monthly or other suitable dosing regimen.

In certain embodiments, methods disclosed herein can treat and/or prevent cancer in a subject in need wherein the subject has a subject determined to have a deletion signal according to the methods disclosed herein. In some embodiments, methods of treatment disclosed herein can impair tumor growth compared to tumor growth in an untreated subject with identical disease condition and predicted outcome. In some embodiments, tumor growth can be stopped following treatments according to the methods disclosed herein. In other embodiments, tumor growth can be impaired at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other words, tumors in subject treated according to the methods disclosed herein grow at least 5% less (or more as described above) when compared to an untreated subject with identical disease condition and predicted outcome. In some embodiments, tumor growth can be impaired at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, tumor growth can be impaired at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

In some embodiments, treatment of tumors according to the methods disclosed herein can result in a shrinking of a tumor in comparison to the starting size of the tumor. In some embodiments, tumor shrinking is at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the tumor is completely gone after treatment) compared to the starting size of the tumor.

In various embodiments, treatments administered according to the methods disclosed herein can improve patient life expectancy compared to the cancer life expectancy of an untreated subject with identical disease condition and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

In some embodiments, a subject to be treated by any of the methods herein can present with one or more cancerous solid tumors, metastatic nodes, of a combination thereof. In some embodiments, a subject herein can have a cancerous tumor cell source that can be less than about 0.2 cm³ to at least about 20 cm³ or greater, at least about 2 cm³ to at least about 18 cm³ or greater, at least about 3 cm³ to at least about 15 cm³ or greater, at least about 4 cm³ to at least about 12 cm³ or greater, at least about 5 cm³ to at least about 10 cm³ or greater, or at least about 6 cm³ to at least about 8 cm³ or greater.

In some embodiments, any of the methods disclosed herein can further include monitoring occurrence of one or more adverse effects in the subject having a deletion signal as determined according to the methods disclosed herein. Exemplary adverse effects include, but are not limited to, hepatic impairment, hematologic toxicity, neurologic toxicity, cutaneous toxicity, gastrointestinal toxicity, or a combination thereof. When one or more adverse effects are observed, the method disclosed herein can further include reducing or increasing the dose of one or more of the PPAR inhibitors, the dose of one or more anticancer drugs (e.g., platinum-based chemotherapeutics) or both depending on the adverse effect or effects in the subject. For example, when a moderate to severe hepatic impairment is observed in a subject after treatment, compositions of use to treat the subject can be reduced in concentration or frequency of dosing with one or more disclosed compounds (e.g., PARP inhibitors) and/or the dose or frequency of the platinum-based chemotherapeutic can be adjusted (e.g., cisplatin) or a combination thereof.

### III. Devices

The present invention further provides deceives for enabling one or more embodiments as described above. In some embodiments, methods disclosed herein may be practiced on computer devices including, but not limited to, a desktop computer, laptop computer, tablet computer, server (e.g., a cloud accessible server), or wireless handheld device. In some embodiments, methods disclosed herein may be practiced on a special purpose computer or data processor, such as application-specific integrated circuits (ASIC), field-programmable gate arrays (FPGA), graphics processing units (GPU), many core processors, and the like. In some aspects, processing units of the devices herein may comprise a central processing unit (“CPU”), a CPU-type processing unit, a GPU-type processing unit, a field-programmable gate array (FPGA), another class of digital signal processor (DSP), or other hardware logic components that can be driven by a CPU. In some embodiments, computer devices and/or data processors herein may be specifically programmed, configured, or constructed to perform one or more of the methods disclosed herein. In some embodiments, methods herein may be performed exclusively on a single device. In some other embodiments, methods herein may be performed in distributed computing environments shared among disparate processing devices, which may be linked through a communications network such as a Local Area Network (LAN), Wide Area Network (WAN), or the internet. In some embodiments, methods performed on devices herein may comprise software assisted by a host (e.g., PC, server, cluster or cloud computing, with cloud and/or cluster storage.)

In some embodiments, methods disclosed herein may be implemented as a computer-readable/useable medium that may include a computer program code to enable one or more computer devices to implement each of the various process steps in a method in accordance with the present disclosure. In some aspects, where more than computer devices perform the entire operation, the computer devices may be networked to distribute the various steps of the operation. It is understood that the terms computer-readable medium or computer useable medium comprises one or more of any type of physical embodiment of the program code. In some aspects, a computer-readable/useable medium can comprise program code embodied on one or more portable storage articles of manufacture (e.g. an optical disc, a magnetic disk, a tape, etc.), on one or more data storage portioned of a computing device, such as memory associated with a computer and/or a storage system.

In some embodiments, provided herein is a computer-implemented method of diagnosing or prognosing a subject with a disorder and/or a condition wherein the subject has not been diagnosed previously, is not suspected of having the disorder and/or the condition, or is suspected of having the disorder and/or the condition. In some embodiments, provided herein is a computer-implemented method of characterizing the stage and/or severity of a disorder and/or a condition wherein the subject has not been diagnosed previously, is not suspected of having the disorder and/or the condition, is suspected of having the disorder and/or the condition, or has been diagnosed with the disorder and/or the condition previously. In some embodiments, there is provided a computer-implemented method of diagnosing or prognosing a subject with cancer or suspected of having cancer comprising: receiving, at least one processor, data reflecting cancer DNA sequencing data from a cancer sample comprising cancer cells from the subject; and determining, at the at least one processor, a risk level. The data reflecting the cancer DNA sequencing data is obtained by first mapping the sequencing data to a genome, identifying deletions in high-complexity sequence context, determining a deletion signal for the DNA-containing sample, wherein the deletion signal comprises a distribution of non-clonal or subclonal deletions and microhomology patterns of DNA sequences flanking sites of mapped deletions in the genome of the subject or tissue sample thereof, decomposing the deletion signal into classes such that deletions due to imperfect DNA repair can be separated from deletions resulting from systematic effects such as presence of personal deletion variants and false positive deletions arising from sample preparation, sequencing, and analysis; and quantifying the deletions resulting from imperfect DNA repair with mixture modeling to produce a quantified deletion distribution. Then, at the at least one processor, the subject is assigned a risk level associated with a patient outcome, wherein a relatively higher risk level is associated with a higher deletion signal and a relatively lower risk level is associated with a lower higher deletion signal.

In some embodiments, there is provided a computer-implemented method of diagnosing or prognosing a subject with cancer or suspected of having cancer comprising: receiving, at least one processor, data reflecting cancer DNA sequencing data from a cancer sample comprising cancer cells from the subject; determining, at the at least one processor, the subclonal populations present in the sample; constructing, at the at least one processor, a phylogenetic map of the subclonal populations; assigning, at the at least one processor, to the subject a risk level associated with a better or worse patient outcome; wherein a relatively higher risk level is associated with a higher level of evolution and number of subclonal populations, and a relatively lower risk level is associated with a lower level of evolution and number of subclonal populations.

In some embodiments, there may be provided a computer program product for use in conjunction with a general-purpose computer having a processor and a memory connected to the processor, the computer program product comprising a computer readable storage medium having a computer mechanism encoded thereon, wherein the computer program mechanism may be loaded into the memory of the computer and cause the computer to carry out the method described herein. In some aspects, there may be provided a computer readable medium having stored thereon a data structure for storing the computer program product described herein.

As used herein, “processor” may be any type of processor, such as, for example, any type of general-purpose microprocessor or microcontroller (e.g., an Intel™ x86, PowerPC™, ARM™ processor, or the like), a digital signal processing (DSP) processor, an integrated circuit, a field programmable gate array (FPGA), a Graphical Processing Unit (GPU) or any combination thereof.

As used herein “memory” may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), or the like. Portions of memory may be organized using a conventional file system, controlled and administered by an operating system governing overall operation of a device.

As used herein, “computer readable storage medium” (also referred to as a machine-readable medium, a processor-readable medium, or a computer usable medium having a computer-readable program code embodied therein) is a medium capable of storing data in a format readable by a computer or machine. The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The computer readable storage medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the computer readable storage medium. The instructions stored on the computer readable storage medium can be executed by a processor or other suitable processing device and can interface with circuitry to perform the described tasks.

As used herein, “data structure” is a particular way of organizing data in a computer so that it can be used efficiently. Data structures can implement one or more particular abstract data types (ADT), which specify the operations that can be performed on a data structure and the computational complexity of those operations. In comparison, a data structure is a concrete implementation of the specification provided by an ADT.

### IV. Kits

The present invention further provides kits for genotyping a sample obtained from a subject, the kit comprising in a container, a means to collect genomic material from the subject, and/or a nucleic acid molecule, an oligo, a peptide, a probe, an antibody, or a combination thereof designed for determining the deletion signal as disclosed herein. Kits disclosed herein may also contain other components such as buffers, reagents, and the like needed to obtain a genetic expression profile of a subject as disclosed herein.

In some embodiments, kits herein may contain any of the devices disclosed herein. In some aspects, kits may further include instructions on how to collect a sample collected from a subject, submit genomic sequence data to any of the data mining methods disclosed herein, how to administer a cancer treatment according to any of the methods disclosed herein, and/or how to operate any of the devices disclosed herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

FIG. 1 depicts a method 100 to detect and classify non-clonal or low clonality deletions that map to high-complexity regions of the genome using sequencing with synchronized and amplified readout. As shown in FIG. 1, sequencing data is provided at 102. In at least some instances the sequencing data may be obtained by sequencing by synthesis. A reference genome may be provided at 104. If the reference genome is used at 104, then the sequencing data is mapped to the reference genome using available mappers at 106. The reference genome provided at 104 may be corrected by comparative genome assembly to obtain a personal genome at 108. For example, a personal genome may be assembled to search for deletions in specific special sequences such as some types of repetitive sequences, such as mitochondria and centromeric repeats, rather than for the entire genome. At 110, the sequencing data may be mapped to the personal genome using available mappers.

At 112 of method 100 depicted in FIG. 1, the mapped reads obtained from 106 or 110 are assessed at 114 to determine if the mapping is high quality. The mapping quality index used here is different from what may be used by standard mappers. First, the length of the deletion has minimal impact on the mapping quality index. Instead, the quality index is upweighted for deletion-containing sequences that map well to the reference genome, i.e. are quite similar on both sides of the deletion (e.g., 95%+ identical to the best match), while substitutions with high Q-values at their positions and multiple indels are downweighed. If the mapped reads from 112 are not determined to be high quality at 114, the reads are discarded at 172. If the mapped reads from 112 are determined to be high quality at 114, the mapped reads are retained at 116 to undergo additional filtering and processing.

At 118, the mapped reads retained at 116 are assessed to determine whether portions of the mapped reads are unmapped or have low Q-values at 118. Deletions cannot be in the low Q-value part of the read. Therefore, deletions in reads are checked using this filter such that a read with a deletion is rejected if the deletion is in a low Q-value region. If reads are unmapped or have low Q-values or have a deletion in a low Q-value region, those reads are discarded at 172. The reads passing the filter in 118 are retained at 120. At 122, the reads retained in 120 are assessed to determine whether they match the pangenome. In this filter, deletions that appear in the pangenome (derived from other genomic data sets) are rejected. The data sets contributing to the pangenome could be genomic data from other human genomes or even from other species, e.g. chimpanzee. The deletions found in the pangenome are rejected even if they are observed at the subclonal level in sequencing data, because their presence in data mapped to the pangenome creates the possibility that the analyzed sequencing read results from a DNA sample obtained from different person. Such contaminations can be introduced by so-called index hopping during sequencing when samples are barcoded or by introducing contamination during the sequencing library preparation. In at least some instances, deletion mapping to the pangenome may include positional sliding. Deletions that map to the pangenome after positional sliding are rejected because substitution errors introduced during sequencing may result in such positional sliding. Reads with deletions close to the read ends are rejected. Reads that match the pangenome are discarded at 172. Reads that pass the filter are retained at 124 for further filtering and processing.

At 126, the mapped reads retained at 124 are assessed to determine whether deletions are in repetitive regions. The first test for repetitive environment checks whether the mapped deletion results in removing tandem repeats. The test includes also approximate tandem repeats (e.g. 90% of identity) and partial removal. Subsequent tests are for low complexity genomic context of a deletion. For example, low complexity analysis may be performed by calculating entropy of kmer distributions for kmers of length 1-5, within the range of ˜60 bp to the left and to the right (˜120 bp total). In other cases, different parameters may be used in the filter, i.e. different functions than entropy, different sizes of sequence regions, and different size of kmers. If a sequencing read is determined to have a repetitive environment, the read is discarded at 172. If a read passes the filter by not having a repetitive environment, the read is retained at 128 for further filtering and processing.

At 130, the mapped reads retained at 128 are assessed to determine if the read comprises a not proper paired end read by analyzing the length of inserts. Only paired-end reads corresponding to the expected insert length are accepted for subsequent analysis. Overlap in read pairs is acceptable. If overlap is present, then consensus sequence resulting from the overlapping between reads (“overlap-seq”) needs to be remapped. If the read comprises a not proper paired end read, the read is discarded at 172. If the read passes the filter, the read is retained at 132 for further filtering and processing.

At 134, the mapped reads retained at 132 are assessed to determine if the read comprises excessive sequencing errors. In particular, reads with multiple substitutions or indel errors are discarded at 172. However, if the substitution or indel corresponds to a personal variant, it is not considered a sequencing error. If the reads are not determined to have excessive sequencing errors, they are retained at 136 for further filtering and processing.

In at least some instances, paired-end sequencing may be used. In paired-end sequencing, a piece of DNA is sequenced from both ends in two sequencing reactions. The result of the first reaction is termed “Read 1” and the result from the second reaction is termed “Read 2.” Read 1 and Read 2 may have the same or different lengths. In some instances, there may also be a “Read 3” when the barcodes introduced in sequencing constructs are sequenced separately. Read 3 usually has a much shorter length (e.g., 8 bp). In paired-end sequencing, a piece of DNA may first be amplified and generate the polony that after sequencing results in Read 1. The same polony is then sequenced from the other end, but this may be performed after additional cycles of synthesis between sequencing Read 1 and the start of sequencing for Read 2. Read 1 is read first, Read 3 is usually read second, and Read 2 is read after Read 3. There may be a “Read 4” as well if more than one index is sequenced.

At 138, the mapped reads retained at 136 are assessed to determine if the read comprises short (e.g., 1-4 bp) indels. During sample preparation, the DNA repair step can generate a significant number of such short deletions that would contribute false positive signal to the somatic deletion signal. Such false positive short deletions are overrepresented in Read 2 (R2) compared to Read 1 (R1), and also have strong positional dependence with excess towards the start of the Read 2. This effect results in false positives also for longer deletions, but longer deletions are statistically less frequent, so the statistical reasoning is more reliable concerning the presence of this effect for short deletions. Therefore, even if these short deletions may not be part of the signal of interest, they provide technical validation. If the mapped reads are determined to comprise short indels, histograms of indels may be determined for R1 and R2 at 140. In some instances, the reads having short indels may be discarded at 172. If the reads pass the filter at 138 they are retained at 142 for further filtering and processing.

At 144, the mapped reads retained at 142 are assessed to determine if the read has a deletion ≥ to 5 base pairs (bp). If the read does not have a deletion ≥ to 5 bp then the read is discarded at 172. If the read does have a deletion ≥ to 5 bp, then the read is retained at 146 for further filtering and processing. At 148, the mapped read retained at 146 is assessed to determine if it comprises a deletion close to the read border. If it does, the read is discarded at 172. If the read retained at 146 does not comprise a deletion close to the read border, the read is retained at 150. At 152, histograms of deletions for R1 and R2 are generated. At 154, it is determined, based on the histograms generated at 152, whether there is an excess of deletions in R2. If there is, R2 is discarded at 156 and only R1 is retained at 158. In cases of overlap-seq, the distance criterion from the 3′ end of the insert is used. If there is not an excess of deletions in R2, the reads are retained in 168.

At 160, the central result of the method is determined. In particular, histograms of microhomology are calculated for reads with some deletion range length, e.g. 10-50 bp. The microhomology histograms are calculated based on three contributors: (1) background, (2) signal of interest, and (3) hybridization events (could be due to DNA repair in sample preparation, PCR amplification, or may be introduced during polony amplification on the flow cell). The background has a strong power law dependence on the length of microhomology. The signal of interest has a shoulder or peak around 3-4 bp of microhomology. Hybridization events have a shoulder that extends above six bp of microhomology. DNA repair during sequencing library preparation may also create a completely different type of signal where R1 and R2 start with an identical sequence and R1 maps to the genome and R2 maps to the genome except for the part of R2 matching R1. The matching between R1 and R2 does not consider sequence complementarity, but compares sequences of raw reads. However, complementarity rules are used when R1 and R2 are mapped together to the reference genome. Therefore, at 170, the reads retained at 158 and 168 may be validated by determining whether R1/R2 start with the same sequence. The presence of this effect is an indicator of problems with DNA repair during library preparation and these problems may correlate with an excessive number of false positive deletions in R2.

At 162, a more elaborate analysis than the microhomology histograms is generated. In particular, ROC curve analysis is performed based on the microhomology histograms calculated at 160 where all cutoffs are optimized to separate the signals. Finally, a predictor is determined at 164 based on the ROC curve analysis. Correlation with phenotypic/genotypic effects may also be determined at 166 based on the ROC curve analysis.

### Example 2

Assumptions: From population genetics, it is known that deletions from 10 to 50 bp happen once per 10¹⁰ bp per generation. Assuming 50% negative filtering and a next generation sequencing dataset having 30× coverage for human genome, one can expect to detect 5 somatic deletions in germline tissues. Higher level of somatic deletions is expected in fast dividing cells so a low false positive rate would be needed to accurately detect somatic (subclonal and non-clonal) deletions.

Assessment with high-complexity genome: The high complexity genome of Pedobacter heparinus having 43% GC, a GC content comparable with human genome, was used in initial analysis. 8.5 Gbp of Pedobacter heparinus sequencing data obtained from PCR-free sequencing library was analyzed. Sequencing reads were mapped with Bowtie 2. The aligned reads were filtered according to the methods presented on the flow chart depicted in FIG. 1. 9 somatic deletions longer than 10 bp were detected in 8.5 Gbp of sequencing data. The result indicates that the false positive rate of around 10⁻⁹ is required for similar analysis of high-complexity regions of human genome.

The same methods (see FIG. 1) were applied to high quality, human WGS dataset (ERP010096). In this dataset, 204 somatic deletions longer than 10 bp were identified, with 41 of them mapping to Alu and LINE elements. The biological background plus the false positive rate was assessed to be lower than 4×10⁻⁹ for this high quality dataset.

### Example 3

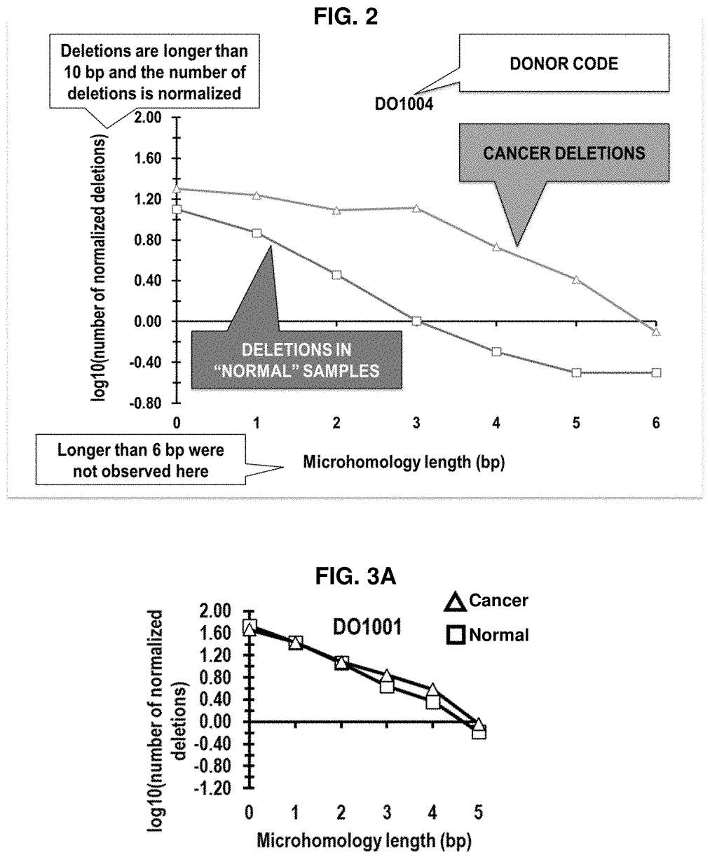

Whole genome sequencing (WGS) data sets from 117 donors were obtained from the ICGC database. WGS was performed using Illumina instruments which use an amplified fluorescence signal for sequencing. Sequencing data were mapped with Bowtie 2 and the mapped sequencing reads were then subjected to data filtering according to the flow chart depicted in FIG. 1. After filtering a very small set of deletions was left within which the microhomology patterns at deletion sites was analyzed. The number of deletions with different microhomology lengths, for both cancer tissue sample and matching blood sample for each donor was counted and results were plotted for comparison. An example of the plot showing the number of deletions with microhomology length from 0 to 6 bp at deletion sites, for both normal sample and cancer sample from a single donor is shown in FIG. 2.

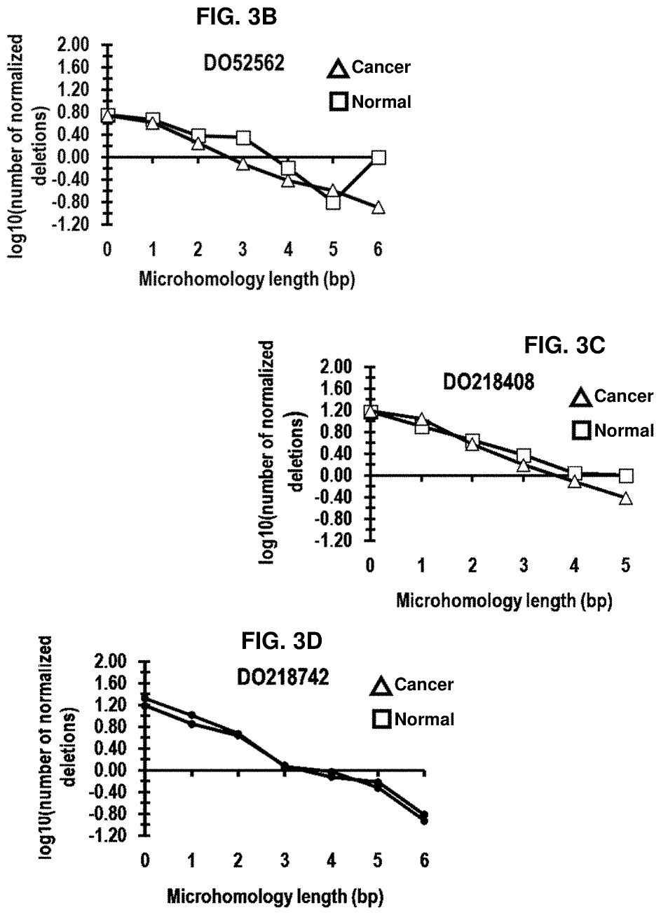

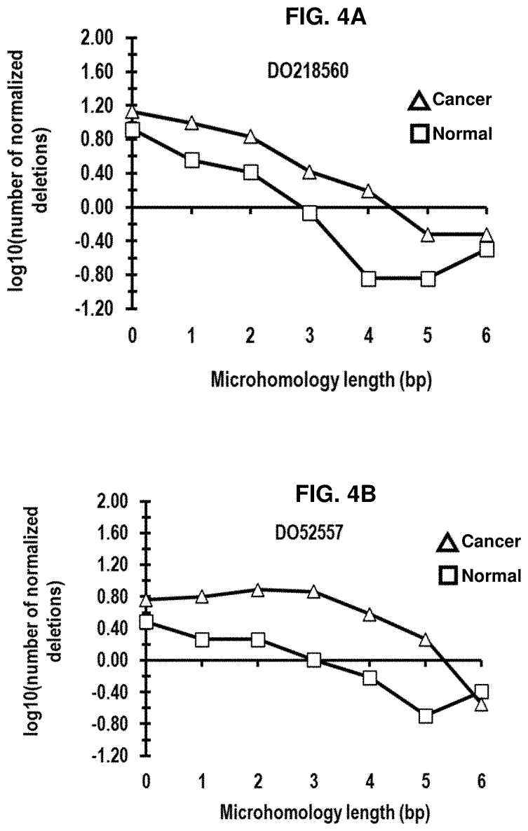

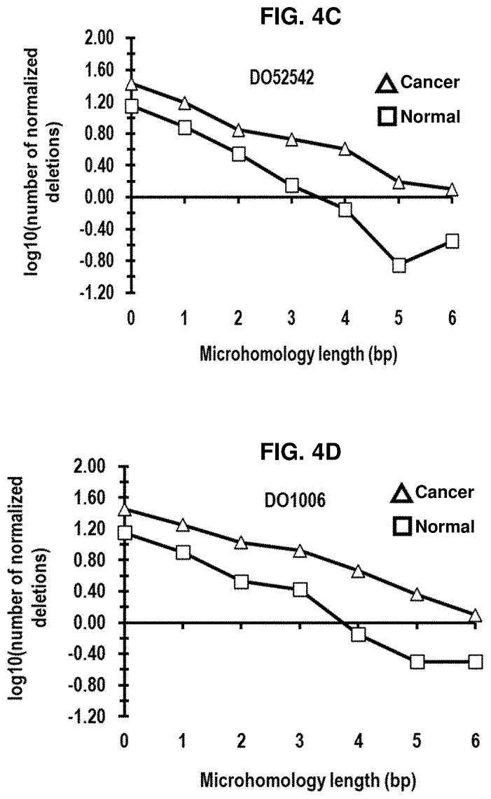

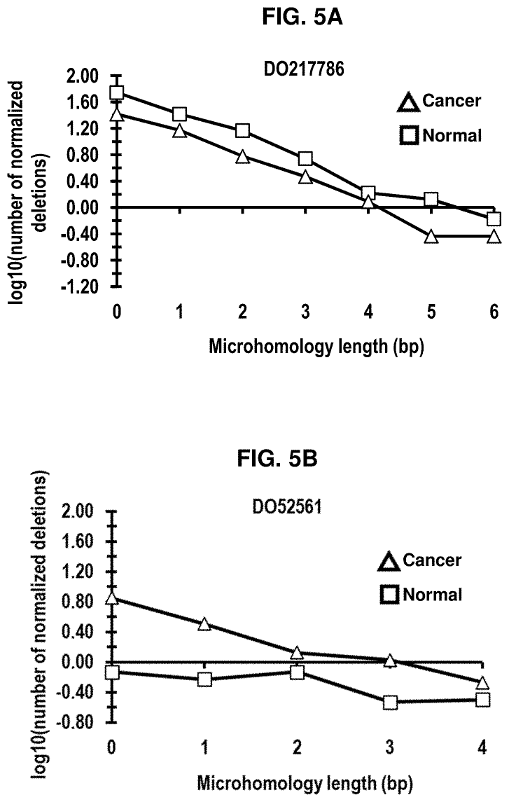

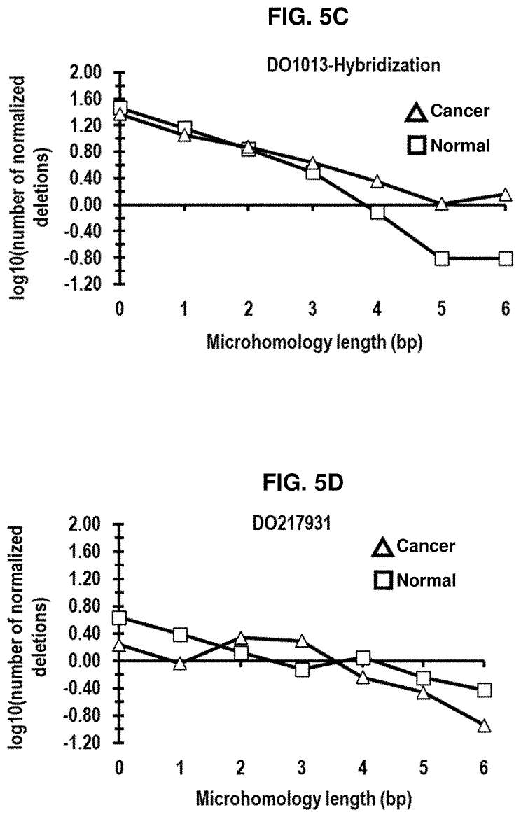

FIGS. 3A-3D show distributions of deletions with microhomologies of length from 0 to 6 bp for representative donor samples. Although deletion signals were detectable there was no difference between cancer and normal samples. FIGS. 4A-4D show distributions of deletions (deletion signals) with microhomologies of length 0 to 6 bp at deletion sites for representative donor samples. The plots show significant difference in levels of deletion signals between signals from normal and cancer samples. Such signals are expected for samples where there was defective HRR redirecting the DNA repair to error-prone mechanisms, and where the process of obtaining sample, preparing sequencing library, and sequencing is well controlled. FIGS. 5A-5D show distribution of deletion signals with microhomologies of length between 0 to 6 bp for representative donor samples, where deletion signals are plotted for normal and cancer samples together. These distributions illustrate effects arising from sources other than defective HRR that one can encounter in data analysis of deletion signals. FIG. 5A, shows that DNA of the control sample has more deletions than DNA of the cancer sample. This was observed a few times in analyzed data and was ascribed to biological differences resulting in purifying selection in the cancer sample or presence of other cancers affecting the control sample. FIG. 5B shows the difference between deletion signals for cancer and normal samples. The interesting feature is very low level of background of somatic deletions, only 25 deletions in the control sample. This figure shows that for well done experiments even such a low signal can be measured. FIG. 5C depicts the possibility of artifacts arising from hybridization during sample preparation or sequencing for cancer sample. Alternatively, the cancer sample could be affected by the process that results in excess of non-clonal deletions with longer microhomologies at the deletion sites. FIG. 5D depicts lack of difference in deletion signals between normal and cancer samples and also very low count of subclonal deletions.

Of the 117 donors analyzed, 27 shown clear difference in deletion signals between cancer and normal samples. Out of these 27 donors, 12 had BRCA1/2 mutations. 90 donors out of the 117 analyzed, did not show a difference in deletion signal between cancer and normal samples, and 15 of those donors had BRCA1/2 mutations.

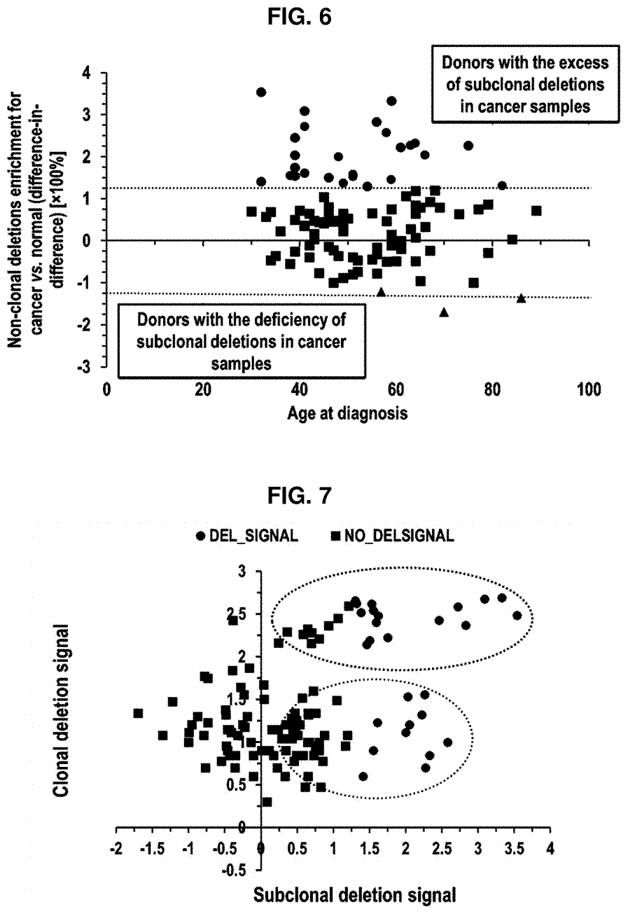

The correlation between age of the donor and the differences in deletional signals in normal and cancer samples were analyzed and is shown in FIG. 6. Each symbol represents a rough quantification (on the y axis) of the difference in deletional signals, defined as the integrated (summed) differences between the logarithms of the number of deletions for cancer and normal plots. The vertical scale was derived from logarithmic scale, so the horizontal dashed lines represent a two-fold difference from the lack of difference. Microarray data are also presented in similar way, with a factor of two representing a significant change. The orange dots represent the donors for which the difference in deletion signals exceeded 2-fold difference. The blue squares represent no difference, spurious difference, or weak difference, and the green triangles show negative difference i.e. normal samples have more deletion signal than the cancer samples.

FIG. 6 shows that there was no age dependence on deletion signals in the analyzed data sets. The method followed a modified difference-in-difference analysis to analyze the difference in decay of the deletion signals between cancer and normal samples. No difference between cancer and normal samples means that the number of deletions with a given microhomology length would be similar for both samples. The change on y axis represented how much more or less [%] subclonal deletions were present. The blue line represents an arbitrary cutoff in data analysis. The proper statistical cutoff can be established with the analysis of more data sets. Three samples in which normal samples had significantly more subclonal deletions than cancer samples were observed but were not sufficient to do any in depth analysis in this example. However, these differences were likely not a mistake in deposition.

For each donor the magnitude of difference in deletion signals between normal and cancer samples was plotted (x-axis) against log10 of the number of clonal deletions longer than 10 bp and shorter than 100 bp (FIG. 7) for the same donor in cancer samples. A deletion was considered clonal if it appeared more than 5 times in the final data. The data for the clonal deletion count was obtained from ICGC. It was observed that the including the difference in the subclonal deletion count in the analysis resulted in the separation of donors into clusters with different combinations of clonal and subclonal deletions. The points representing donors are arbitrarily colored according to the level of the difference in the deletion signals between normal and cancer samples (see FIG. 6). The orange dots represent donors with significant differences in deletion signals (FIG. 6) whereas the blue square represent donors with differences in deletion signals that were considered not significant. The addition of the clonal signal as the second coordinate revealed two groups of donors with high and low levels of clonal deletions. The differential deletion signal is present in both these clusters, although more frequently in the cluster with high level of clonal deletions. Low level of clonal deletions and low level of difference between cancer and normal samples in subclonal deletion signal indicates that the mutator phenotype is not involved. The bottom right cluster where there is high signal from the subclonal component but low signal from the clonal component corresponds to the presence of a mutator that is either responsible for clonal expansion or else originated around the same time. The top cluster corresponds to the situation where the mutator originated significantly prior to the last clonal expansion. Therefore, methods herein provide an approach to determine whether a mutator was directly responsible for a clonal expansion or not.

Secondly, the presence of a mutator was detected, which was actionable, even in the absence of clonal mutations. At the moment, clonal mutations are the only way to identify a mutator—or else by analyzing specific genes being mutated (like BRCA). But here, the presence of a mutator was observed both in the presence and absence of a BRCA mutation. These data demonstrate that it is possible that a larger class of people could be treated with PARP inhibitors based on identification of deletion signals using the methods herein.

In the scatter plot of FIG. 7, the orange dots are split roughly equally into two clusters, whereas the blue squares are also split into two rough clusters, but in an 8 to 1 ratio. The fact that the orange dots have a different distribution than the blue squares supports the correlation between clonal and subclonal mutational processes. However, depending on the timing of the origin of the mutator phenotype compared to the clonal expansion, the correlation between clonal and subclonal components were only partial. Having blue squares in this orange dot-heavy cluster was an indication that there was likely a recent clonal expansion in the cancer belonging to the blue square donors, but that the mutator was old. The orange dots on the left showed the opposite—an old clonal expansion with a mutator that appeared around the time of the expansion.

### Example 4

Whole genome sequencing (WGS) was performed on DNA isolated from HCC1395BL and HCC1395 cell lines. HCC1395BL is a human B lymphoblastoid cell line initiated by Epstein-Barr virus (EBV) transformation of peripheral blood lymphocytes obtained from the same patient as the breast carcinoma cell line HCC1395. Accordingly, HCC1395BL cells served as a control or normal sample for the HCC1395 cells, which is BRCA1 homozygous, triple negative, derived from primary ductal carcinoma.

Using DNA isolated from these two cell lines, two combinations of DNA fragmentation (Kapa and Nextera) and two types of sequencers (HiSeq2500 and HiSeq4000) were used. HiSeq2500 uses non-patterned cells and therefore it was expected that it is less prone to the formation of hybrids compared to HiSeq4000 which uses a patterned flow cell. Accordingly, Nextera to HiSeq2500, Nextera to HiSeq4000, Kapa to HiSeq2500, and Kapa to HiSeq4000 were tested. Over two lanes, sixteen data sets were processed.

Sequencing data were aligned to reference genome with Bowtie 2 and the mapped reads were subjected to data filtering according to the process depicted in FIG. 1. The filtering included removal of tandem repeats, deletions less than or equal to 10 base pairs, approximate tandem repeats, locally repetitive sequences, globally repetitive sequences, deletions too close to sequencing read ends, read pairs that were discordantly mapped, reads with too many substitution errors, and deletions observed elsewhere. Then population polymorphisms were removed. Population variants were removed using three reference sets of data on personal polymorphisms: 1) the GNOMAD database, 2) the personal polymorphism set calculated from the sequencing of the HCC1395BL and HCC1395 cells herein, and (3) the personal polymorphism set calculated for all other data sets that we processed.

The filtering also included removal of repetitive regions from the data analysis. Repetitive regions generate problems in library and sequencing process that results in sequencing errors mimicking deletions. Such problems are particularly pronounced if DNA is fragmented or incompletely replicated in overloaded PCR.

Sequencing data were then filtered to remove hybrids and deletions that were shorter than 10 bp.

Two examples of the results from the data mining methods performed on HCC1395BL and HCC1395 cell sequences are provided in Table 3 and Table 4 below.

TABLE 3
Sequencing method: HiSeq2500
Method of library formation used: Nextera

| Data Mining Steps | Results |
|---|---|
| STEP 1: Read pairs | 199,203,220 read pairs [100%] |
| STEP 2: After alignment with Bowtie 2 | 122,629,647 read pairs [61.56%] |
| STEP 3: Filters (selected) | |
| remove reads 1000+ bp apart | 4,414,894 read pairs removed |
| remove reads with bad MAPQ | 1,388,262 read pairs removed |
| remove invalid TLEN | 16,813 read pairs removed |
| remove hard clipped reads | 14,340,757 read pairs removed |
| remove reads on separate chromosomes | 398,600 read pairs removed |
| remove reads with unidirectional | 135,761 read pairs removed |
| mapping | |
| remove population polymorphisms | 931,825 read pairs removed |
| remove for repetitiveness | 1,027,894 read pairs removed |
| bad quality counter | 52,741 read pairs removed |
| remove hybrids | 8 read pairs removed |
| remove deletions | 5,600 read pairs removed |
| remove reads with low complexity | 377 read pairs removed |
| Non-clonal and subclonal deletions used | 292 deletions detected |
| in microhomology analysis | |

TABLE 4
Sequencer: HiSeq2500
Method of library preparation: KAPA

| Data Mining Steps | Results |
|---|---|
| STEP 1: Read pairs | 57,336,298 read pairs [100%] |
| STEP 2: After alignment with Bowtie 2 | 36,141,684 read pairs [63.03%] |
| STEP 3: Filters (selected) | |
| remove reads 1000+ bp apart | 2,101,462 read pairs removed |
| remove reads with bad MAPQ | 886,869 read pairs removed |
| remove invalid TLEN | 10,640 read pairs removed |
| remove hard clipped reads | 8,803,546 read pairs removed |
| remove reads on separate chromosomes | 394,076 read pairs removed |
| remove reads with unidirectional | 59,855 read pairs removed |
| mapping | |
| remove population polymorphisms | 520,207 read pairs removed |
| remove for repetitiveness | 583,224 read pairs removed |
| bad quality counter | 52,741 read pairs removed |
| remove hybrids | 4 read pairs removed |
| remove deletions | 4000 read pairs removed |
| remove reads with low complexity | 291 read pairs removed |
| Non-clonal and subclonal deletions used | 240 deletions detected |
| in microhomology analysis | |

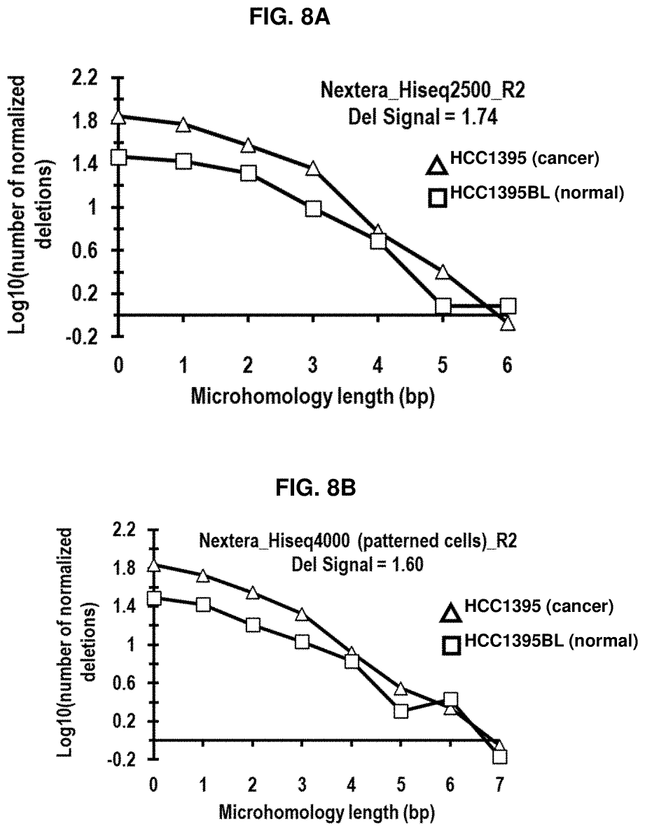

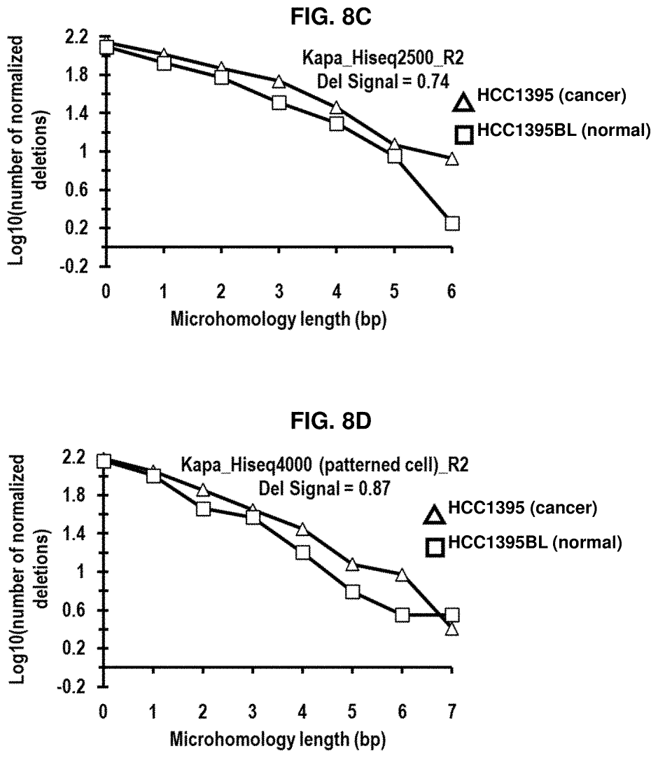

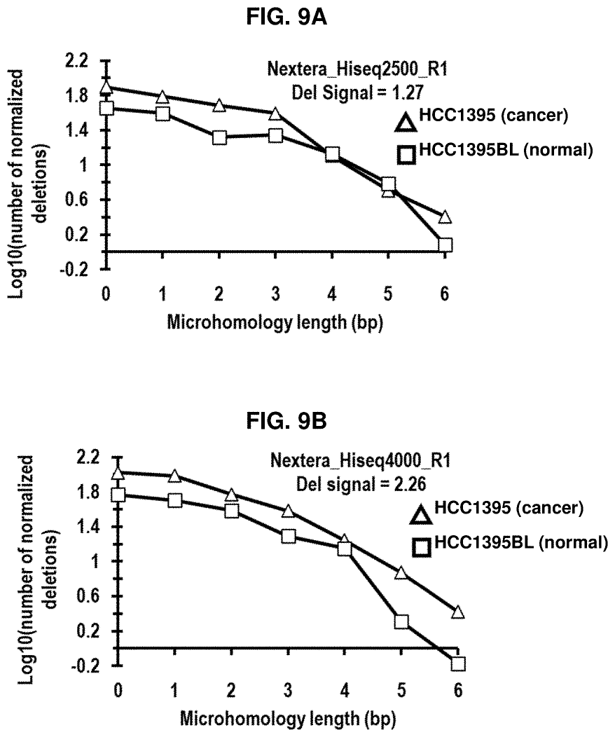

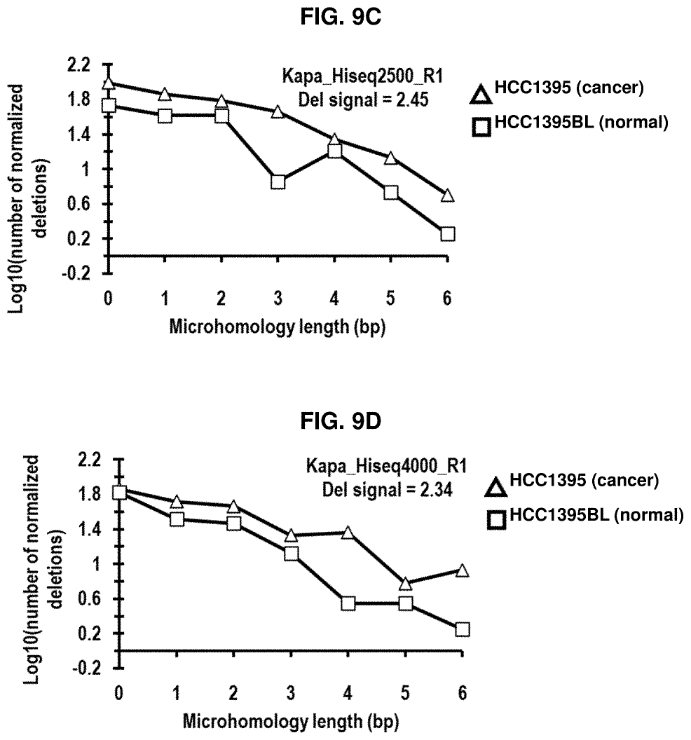

FIGS. 8A-8D and FIGS. 9A-9D show deletion signals from data mining methods performed on sequencing data from HCC1395BL and HCC1395 cell lines. These tests were performed to establish whether existing sequencing approaches are sensitive enough to detect deletional signals described in the invention. The experiment informed how artifacts detected on sequencing read 2 (R2) depend on the method of sequencing library preparation (Nextera that is based on tagmentation vs Kapa that involved PCR amplification), on sequencing hardware (4-color readout with flow cells with randomly distributed polonies as in HiSeq2500 vs 2-color readout with patterned flow cells as in HiSeq4000). Sequencing libraries prepared with Kapa kit showed higher background and a little separation between deletion signals from cancer and normal samples (FIGS. 8C-8D). Nextera libraries had a lower background and a significant separation between deletion signals for cancer and normal samples (FIGS. 8A-8B) for R2. Analyzing just sequencing read R1 allows to achieve the separation of the normal and the cancer deletion signals also for Kapa library and the signal appeared in all four plots (FIGS. 9A-9D). A difference in deletion signals between normal and cancer cell lines was observed for the microhomology patterns at deletion sites of length between 0 to 6 bp. (FIGS. 9A-9D). It was determined that PCR amplification may cause these differences, while the type of flow cell used and the instrument readout type did not affect the deletion signals.

1. A method comprising:
providing sequence data, comprising a plurality of sequencing reads, for a DNA-containing sample of a subject, wherein the sequence data is obtained by sequencing by synthesis;
mapping the sequencing reads to a genome;
identifying deletions in high-complexity sequence context;
determining a deletion signal for the DNA-containing sample, wherein the deletion signal comprises a distribution of non-clonal or subclonal deletions and microhomology patterns of DNA sequences flanking sites of mapped deletions in the genome of the subject or tissue sample thereof;
decomposing the deletion signal into classes such that deletions due to imperfect DNA repair can be separated from deletions resulting from systematic effects such as presence of personal deletion variants and false positive deletions arising from sample preparation, sequencing, and analysis; and
quantifying the deletions resulting from imperfect DNA repair with mixture modeling to produce a quantified deletion distribution.

2. The method according to claim 1, further comprising determining, based on the quantified deletion distribution, a clonal profile for the subject, wherein the clonal profile comprises at least one clonal deletion.

3. The method according to claim 1, further comprising determining, based on the quantified deletion distribution, a subclonal profile for the subject, wherein the clonal profile comprises at least one subclonal deletion distinct from one or more clonal deletions.

4. The method according to claim 1, further comprising determining a correlation between the quantified deletion distribution and one or more clonal substitutions.

5. The method according to claim 1, wherein the correlation between the quantified deletion distribution and the one or more clonal substitutions comprises a correlation between the deletion distribution of the at least one subclonal deletion distinct from one or more clonal deletions and one or more patterns of the one or more clonal substitutions.

6. The method according to claim 1, wherein the decomposing comprises using sequence entropy to select high-complexity regions and exponential modeling to filter out the systematic effects.

7. The method according to claim 1, wherein the decomposing comprises determining one or more vector properties based on alignment to a reference genome, the one or more vector properties selected from the group consisting of a microsatellite index, surrounding sequence entropy, an indicator of the presence of a genome-wide repetitive element, distance from the read start and read end, and personal variant determination.

8. The method according to claim 1, wherein the decomposing comprises determining one or more vector properties based on alignment to a reference genome, the one or more vector properties selected from the group consisting of a microsatellite index, surrounding sequence entropy, an indicator of the presence of a genome-wide repetitive element, distance from the read start and read end, and personal variant determination, wherein:
the personal variant determination vector property is determined based on mapping the regions surrounding the putative deletions on all other reads in order to determine whether or not it is a personal variant that mappers failed to recognize in other reads;
the decomposing further comprises generating, based on the one or more vector properties, a receiver-operator characteristic (ROC) curve using exponential modeling;
tensorial blind source decomposition is used to optimize the weights of the receiver-operator characteristics on the ROC curve to achieve optimal isolation of deletions; and/or
further comprising determining a ROC curve cutoff for isolating deletions using standard maximum likelihood reasoning.

9. The method according to claim 1, wherein the deletions result from non-homologous end joining (NHEJ) dsDNA break repair.

10. The method according to claim 1, wherein the decomposing comprises classifying the distributed deletions in the deletion signal based on deletion sequence length and adjacent microhomology patterns.

11. The method according to claim 1, wherein the DNA-containing sample comprises a blood or tissue sample.

12. The method according to claim 1, further comprising obtaining a whole genome sequencing (WGS) data set for the DNA-containing sample of the subject.

13. The method according to claim 1, further comprising determining, based on the quantified deletion distribution, a mutational signature or biomarker corresponding to one or more cancers.

14. The method according to claim 1, further comprising determining, based on the quantified deletion distribution, a mutational signature or biomarker corresponding to one or more cancers, and further comprising modifying or formulating a cancer treatment for the subject based on the quantified deletion distribution or the mutational signature, such as wherein the one or more cancers is a BRCA1 or BRCA2 mutation-positive cancer.

15. The method according to claim 1, further comprising assessing, based on the quantified deletion distribution, the significance of the variants of unknown significance (VUS) in the subject.

16. The method according to claim 1, wherein:
the method is a method of assessing and quantifying imperfect dsDNA break repair;
the method is a method of diagnosing cancer;
the method is a method for assessing the genotoxicity of a therapeutic treatment;
the method is a method for assessing the genotoxicity of a therapeutic cancer treatment;
the method is a method for the monitoring of cancer progression in a subject;
the method is a method for the early detection of cancer; or the method is a method for the prevention or treatment of cancer.

17. The method according to claim 1, wherein the method is a method for the personalization of treatment of cancer in a subject, the method comprising:
determining whether cancer cells in the subject will be sensitive to the administration of a predetermined small molecule.

18. The method according to claim 17, wherein the predetermined small molecule is a poly adenosine diphosphate (ADP) ribose polymerase (PARP) inhibitor.

19. The method according to claim 17, wherein the cancer is a cancer with defects in BRCA1/2 genes.

20. A device comprising:
at least one processor coupled with a non-transitory computer-readable storage medium having stored therein instructions which, when executed by the at least one processor, causes the at least one processor to perform the method, or any elemental step thereof, according to claim 1.
